(19) United States
(12) Patent Application Publication
(10) Pub. No.: US 2026/0131033 A1
(43) Pub. Date: May 14, 2026

(54) RADIOLABELED FRUCTOSE DERIVATIVES FOR MEDICAL IMAGING

(71) Applicant: Yellowbird Diagnostics Inc., Orleans (CA)

(72) Inventors: Adam Jason SHUHENDLER, Ottawa (CA); Mojmir SUCHY, Ottawa (CA); Robert BEN, Ottawa (CA); Thomas CHARLTON, Ottawa (CA); Alexia Margarite KIRBY, Ottawa (CA); Nicholas David CALVERT, Ottawa (CA)

(21) Appl. No.: 19/329,432

(22) Filed: Sep. 15, 2025

Related U.S. Application Data
(63) Continuation of application No. PCT/CA2024/051550, filed on Nov. 22, 2024.
(60) Provisional application No. 63/610,612, filed on Dec. 15, 2023.
(60) Provisional application No. 63/715,119, filed on Nov. 1, 2024.

(51) Int. Cl. A61K 51/04; A61P 25/28; C07B 59/00; C07H 5/02

(52) U.S. Cl. CPC A61K 51/0491; A61P 25/28; C07B 59/005; C07H 5/02; A61K 2123/00

(57) ABSTRACT

There are provided fructose-based radiopharmaceuticals, pharmaceutical compositions comprising same, methods for preparing same, and methods of using same for biomedical imaging, particularly nuclear imaging using PET or PET/CT. (Compound 7)

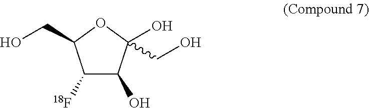 [SMILES: OC[C@H]1OC(O)(CO)[C@@H](O)[C@@H]1[18F]]

### FIELD

The present disclosure relates to the field of biomedical imaging, and in particular radiolabeled derivatives of fructose, their methods of manufacture and their uses.

### BACKGROUND

Positron emission tomography (PET) is a nuclear medicine imaging technique for studying metabolic and physiological processes and tissue microenvironments, and diagnosing or treating diseases including cancer, heart disease, brain abnormalities, and other conditions. PET uses non-toxic radiopharmaceutical agents formed from biologically relevant molecules labelled with positron-emitting radionuclides. Following administration to the body, the radiopharmaceutical agent localizes within the tissue of interest. When the isotope decays, it emits a positron which then annihilates an electron of a nearby atom, producing gamma rays. The PET scanner detects gamma ray photons, thereby producing an image of the tissue for interpretation by a radiologist.

Nuclear medicine images are often combined with computed tomography (CT). Combined PET/CT scanners combine, in a single unit, a PET scanner and an x-ray CT scanner, to acquire sequential images from both devices in the same session, which are combined into a single superposed image. This combination allows alignment of the functional PET imaging, which depicts the spatial distribution of metabolic or biochemical activity, with the anatomic imaging obtained by CT scanning. PET can also be combined with Magnetic Resonance Imaging (PET/MRI). The use of fructose as an energy source (i.e., fructolysis) during the onset and progression of a variety of diseases is a continued area of both fundamental and clinical investigation. Fructose metabolism has been implicated in various diseases, including metabolic disorders, neurodegenerative disorders, cardiac disorders, and cancer, in which inflammation-induced energy crises activate a fructolytic state in the affected tissues.

In the heart, the switch from glycolysis to fructolysis has been identified in cardiac hypertrophy (Mirtschink, P. et al., 2015; Mirtschink, P. et al., 2018) and myocardial infarction (Williams, A. L. et al., 2018), with data supporting a hypoxia-driven activation of this aberrant metabolic program. In the brain, fructolysis is thought to be a putative driver of Alzheimer's disease (Johnson, R. J. et al., 2020), and has been shown to be pro-inflammatory with negative implications following traumatic injury, stroke injury, and psychological health M. S. et al., 2021). The switch from glucose to fructose as energy source may also be a key oncologic driver, promoting the progression of a variety of solid tumors through the concerted transcriptional activation of transport and metabolic machinery (Chen, C. et al., 2022; Liu, H. et al., 2010; Helsey, R. N. et al., 2023; Ward, P. S. et al., 2012; Song, A. et al., 2023). Excessive fructose consumption has also been associated with a hepatic-centered metabolic syndrome thought to drive obesity and diabetes (Hannou, S. A. et al., 2018), and to be a major player in the related cardiovascular (Hannou, S. A. et al., 2018; Kolderup, A. et al., 2015), ocular (Harder, J. M. et al., 2020), and degenerative outcomes (Kolderup, A. et al., 2015).

Fluorodeoxyglucose (FDG) (Pacak, J. et al., J. Chem. Soc. D 1969, 77) and its ¹⁸Fluoro-derivative (¹⁸F-FDG) (Ido, T. et al., J. Label. Compd. Radiopharm. 1978, 14. 175-183) have been widely used in PET and PET-CT imaging due to the higher metabolism of carbohydrates in cancer cells. The success of FDG and ¹⁸F-FDG led to subsequent investigation of other carbohydrates and their possible application as nuclear imaging agents according to their own unique metabolic pathways. Both 1-deoxy-1-fluoro-fructose (1-[¹⁸F]FDF) and 6-deoxy-6-fluoro-fructose (6-[¹⁸F]FDF) were developed as a rational design for hexose transport pathways (Haradahira, T. et al., 1995; Trayner, B. J. et al., 2009; U.S. Pat. No. 8,293,208) and have shown potential for PET imaging of GLUT5 and ketohexokinase-expressing tumors (Wuest, M. et al., 2011).

However, the evaluation of fructolysis in fundamental mechanisms of pathology and its implementation as a diagnostic imaging biomarker has been limited by the lack of a quantitative tracer for imaging-based analysis. C₁- and C₆-fluorinated fructose analogs (such as 1-[¹⁸F]FDF and 6-[¹⁸F]FDF) result in fluorolactate accumulation in vitro, and [¹⁸F]6-FDF displays substantial bone uptake due to metabolic processing in vivo.

There is a need for a quantitative imaging radiotracer for fructolysis that can overcome the limitations of previous fluorinated fructose analogs to provide clinically viable tools for the imaging of fructolysis.

### SUMMARY

An object of the present invention is to provide radiofluorinated fructose derivatives for medical imaging. In accordance with an aspect of the present invention, there is provided compound of Formula (I), or a pharmaceutically acceptable salt thereof:

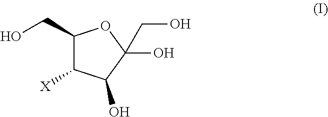

wherein X is ¹⁸F, ¹¹C, ⁷⁵Br, ⁷⁶Br, ⁷⁷Br, ¹²⁴I, ¹³¹I, or ²¹¹At.

In accordance with another aspect of the present invention, there is provided compound of Formula (IIa) or (IIa′), or a pharmaceutically acceptable salt or ester thereof:

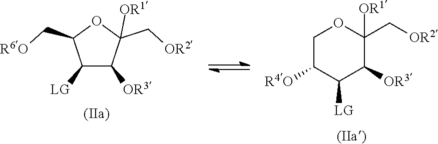

wherein: R^{1′}, R^{2′}, R^{3′}, R⁴ and R^{6′} are independently selected from —C(═O)lower alkyl and —C(═O)Ph; and LG is 4-methylphenylsulfonyl (—OTs), methanesulfonyl (—OMs), trifluoromethansulfonyl (—OTf), 4-nitrophenylsulfonyl (—ONs), phenylsulfonyl, —Br, —Cl, or —I.

In accordance with another aspect of the present invention, there is provided a compound of formula (IIb) or (IIb′), or a pharmaceutically acceptable salt or ester thereof:

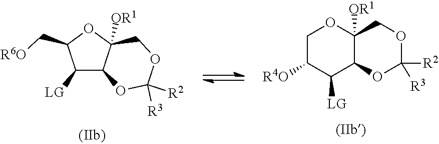

wherein: R¹ is lower alkyl; R² is H and R³ is phenyl, or R² and R³ are each independently lower alkyl; R⁴ and R⁶ are methoxymethyl (MOM), t-butyl (t-Bu), benzyl (Bn), trimethylsilyl (TMS), t-butyldimethylsilyl (TBDMS), or tetrahydropyranyl (THP); and LG is 4-methylphenylsulfonyl (—OTs), methanesulfonyl (—OMs), trifluoromethansulfonyl (—OTf), 4-nitrophenylsulfonyl (—ONs), phenylsulfonyl, —Br, —Cl, or —I

In accordance with another aspect of the present invention, there is provided use of a compound or a composition of the present invention for biomedical imaging of one or more tissues in a subject.

In accordance with another aspect of the present invention, there is provided a method of biomedical imaging, comprising administering the compound or composition of the present invention to a subject, and imaging one or more tissues in the subject using PET, PET/CT, or PET/MRI to measure accumulation of compounds of Formula (I) and metabolites of the compound in the tissue.

In accordance with another aspect of the present invention, there is provided a method for diagnosing and/or monitoring a fructolysis-associated disorder in a subject, comprising administering an effective amount of the compound or composition of the present invention to the subject and imaging one or more tissues in the subject using positron emission tomography (PET) to measure accumulation of compounds of Formula (I) and metabolites of the compound in the one or more tissues.

In accordance with another aspect of the present invention, there is provided a method for diagnosing or monitoring cancer in a subject comprising administering an effective amount of the compound or composition of the present invention to the subject and imaging one or more tissues in the subject using positron emission tomography (PET) to measure accumulation of compounds of Formula I and the metabolites in the one or more tissues.

In accordance with another aspect of the present invention, there is provided a method for monitoring cancer and/or cancer treatment in a subject, comprising: administering an affective amount of the compound or composition of the present invention to the subject, wherein the subject is undergoing medical treatment for cancer; imaging cancer tissue in the subject using positron emission tomography (PET) to measure accumulation of compounds of Formula (I) and metabolites of the compound in the one or more tissues; and comparing the quantity or distribution of the compounds of Formula (I) and metabolites present in the subject with a control quantity or distribution indicative of the effectiveness of the medical treatment.

In accordance with another aspect of the present invention, there is provided a method of treating cancer comprising administering the compound or composition of the present invention, wherein X is ²¹¹At, ¹³¹I, and ⁵⁷Br, to outcompete cancer cells for fructose in GLUT-mediated hexose uptake.

In accordance with another aspect of the present invention, there is provided use of the compound or composition of the present invention for nuclear medicine imaging of fructolysis in a subject.

In accordance with another aspect of the present invention, there is provided a radiopharmaceutical for use in biomedical imaging using PET, PET/CT or PET/MRI comprising the compound or composition of the present invention.

In accordance with another aspect of the present invention, there is provided a method of preparing a compound of Formula (I) comprising the steps of: reacting a precursor compound of Formula (IIa) or (IIa′) or a compound of Formula (IIb) or (IIb′) with a radioactive fluorinated complex to displace the leaving group (LG) and provide a protected [¹⁸F]4-fluorofucose intermediate; and deprotecting the protected [¹⁸F]4-fluorofucose intermediate to yield the compound of Formula (I).

In accordance with another aspect of the present invention, there is provided a method of preparing a compound of Formula (I) comprising: preparing a precursor compound of Formula (IIa) or (IIa′) or a compound of Formula (IIb) or (IIb′); reacting the precursor compound with a carrier for [¹⁸F]F to enable nucleophilic substitution of the leaving group (LG) at C4, to prepare a protected [¹⁸F]4-fluorofucose intermediate; and removing the protecting groups in the protected [¹⁸F]4-fluorofucose intermediate to yield the compound of Formula (I)

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the invention and to show more clearly how it may be carried into effect, reference will now be made by way of example to the accompanying drawings, which illustrate aspects and features according to embodiments of the present invention, and in which:

FIG. 1 is a chromatogram showing the analytical radio-HPLC trace after injection of 98 kBq of [¹⁸F]-4-fluoro-4-deoxyfructose in H₂O.

FIGS. 2A-2C show an overview of fructose metabolism tracing.

FIGS. 3A-3G show results decoding the positional effects of fructose deoxyfluorination on its metabolism in vitro in HepG2 cells by mass spectrometry.

FIGS. 4A-4D show comparative PET/CT imaging in a heterotopic HepG2 xenograft mouse model.

FIGS. 5A-5F show imaging of inflammation in the brain and heart.

FIG. 6 shows TACs in HepG2 xenograft-bearing mice after [¹⁸F]4-FDF (top), [¹⁸F]6-FDF (middle), and [¹⁸F]FDG (bottom) PET/CT imaging. Solid line=mean; Shaded region=standard deviation.

FIG. 7 shows results from image-based dosimetry in Balb/C mice (n=4) using OLINDA software showing organ dose deposition of [¹⁸F]-4-fluoro-4-deoxyfructose (black) and [¹⁸F]-2-fluoro-2-deoxyglucose.

FIG. 8 shows time-course biodistribution of [¹⁸F]-4-fluoro-4-deoxytagatose in Balb/C mice freely fed (top row) or after overnight fasting (bottom row).

FIG. 9 shows TACs for [¹⁸F]-4-fluoro-4-deoxytagatose uptake in Balb/C mice freely fed (left) or after fasting (right) in the indicated tissues. Solid line=mean; Shaded region=standard deviation.

FIG. 10 shows time-course biodistribution of [¹⁸F]-4-fluoro-4-deoxyfructose in Balb/C mice freely fed (top row) or after overnight fasting (bottom row).

FIG. 11 shows TACs for [¹⁸F]-4-fluoro-4-deoxyfructose uptake in Balb/C mice freely fed (left) or after fasting (right) in the indicated tissues. Solid line=mean; Shaded region=standard deviation.

FIG. 12 Shows axial (top row) and sagittal (bottom row) PET/CT images of Balb/C mice receiving [¹⁸F]-4-fluoro-4-deoxyfructose, where the mice were either naïve (left column), or have received a single, closed head concussive impact 48 h prior to imaging (middle and right columns). Arrowhead indicates approximate location and direction of concussive blow.

### DETAILED DESCRIPTION

It is an object of the present invention to ameliorate at least some of the deficiencies present in the prior art. Embodiments of the present technology have been developed based on the inventors' appreciation that there is a need for improved contrast agents for clinical purposes.

The present invention is based on a molecular design strategy based on the catalytic mechanism of aldolase, a key enzyme in fructolysis. Based on aldolase-guided C₄-radiodeoxyfluorination of fructose, a novel radiodeoxyfluorinated fructose analog, [¹⁸F]-4-fluoro-4-deoxyfructose ([¹⁸F]4-FDF), in high molar activity, was designed and synthesized. As described hereinbelow, heavy isotope tracing by mass spectrometry was used to demonstrate that C₄-deoxyfluorination of fructose led to effective trapping as fluorodeoxysorbitol and fluorodeoxyfructose-1-phosphate in vitro, unlike C₁- and C₆-fluorinated analogs that resulted in fluorolactate accumulation. This observation was consistent in vivo, where [¹⁸F]6-FDF displayed substantial bone uptake due to metabolic processing, while [¹⁸F]4-FDF did not. Importantly, [¹⁸F]4-FDF exhibited low radiotracer uptake in healthy brain and heart tissues, known for their high glycolytic activity and background levels of [¹⁸F]FDG uptake. [¹⁸F]4-FDF PET/CT allowed for sensitive mapping of neuro- and cardioinflammatory responses to systemic LPS administration. Thus, [¹⁸F]4-FDF can enable effective radiotracer trapping, overcoming limitations of C₁- and C₆-radioanalogs towards a clinically viable tool for imaging fructolysis in highly glycolytic tissues.

The present disclosure provides fructose derivative compounds suitable for use clinically. Compounds provided herein can allow reliable, rapid, and/or quantitative clinical imaging of fructolysis, which can facilitate early diagnosis, detection and/or monitoring of various disorders. In particular, compounds are advantageous for nuclear imaging, e.g., for positron emission tomography (PET), PET/CT or PET/MRI imaging. Compounds of the disclosure are capable of being retained within a tissue, e.g., a cancer tissue, an inflammatory site, etc., sufficient to emit positrons for detection by nuclear imaging methods such as PET. Without wishing to be limited by theory, compared to previous fluorinated fructose analogs (such as 1-[¹⁸F]FDF and/or 6-[¹⁸F]FDF), compounds of the disclosure provide one or more advantages for imaging fructolysis. For example, they are trapped as fluorodeoxysorbitol and fluorodeoxyfructose-1-phosphate (metabolites along the sorbitol and aldolase pathway) in vitro (unlike C₁- and C₆-fluorinated fructose analogs that resulted in fluorolactate accumulation), leading to high retention/trapping within cells. Compounds of the disclosure halt metabolism at prior to Aldolase catalytic activity and retain the radiofluorine on the molecule, resulting in imaging signal retention where there is high fructose utilization by tissues (i.e., fructolysis). Trapping of compounds of the disclosure upon intracellular uptake and phosphorylation can overcome limitations to fructolysis tracing by C₁- and C₆-radioanalogs and offer a clinically viable tool accessing fructolysis as an imaging diagnostic in tissues with a high baseline glycolytic index.

In certain embodiments, compounds of the disclosure can provide one or more of the following advantages, compared to previous fluorinated fructose analogs (such as 6-[¹⁸F]FDF): reduced loss of ¹⁸F from the compound, as determined for example by measuring bone uptake in mice; and, improved retention of fructose signal in tissues that take up the compound (such as, for example and without limitation, tumor, liver, diseased brain). Moreover, compounds of the disclosure are taken up only in diseased brains (e.g., inflamed brains) and not in healthy brains, providing higher sensitivity to pathology in the brain compared to previous radiopharmaceuticals.

In one aspect, there is provided compound of Formula (I), or a pharmaceutically acceptable salt thereof:

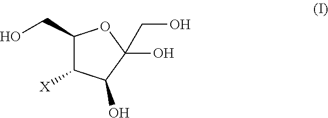

wherein X is any radioisotope suitable for medical imaging. Examples of radioisotopes for use in compounds of the disclosure include, without limitation, ¹⁸F, ¹¹C, ⁷⁵Br, ⁷⁶Br, ⁷⁷Br, ¹²⁴I, ¹³¹I, or ²¹¹At. In a preferred embodiment, X is ¹⁸F.

In one embodiment, the compound is:

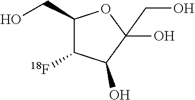 [SMILES: OC[C@H]1OC(O)(CO)[C@@H](O)[C@@H]1[18F]]

### or [¹⁸F]4-FDF (Compound 7).

In another aspect, there is provided a precursor compound of Formula (IIa) or (IIa′), or a pharmaceutically acceptable salt or ester thereof:

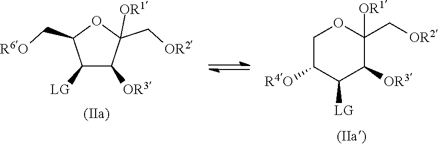

wherein: R^{1′}, R^{2′}, R^{3′}, R^{4′} and R^{6′} are independently selected from —C(═O)lower alkyl and —C(═O)Ph; and LG is 4-methylphenylsulfonyl (—OTs), methanesulfonyl (—OMs), trifluoromethansulfonyl (—OTf), 4-nitrophenylsulfonyl (—ONs), phenylsulfonyl, —Br, —Cl, or —I.

In one embodiment, the precursor compound is

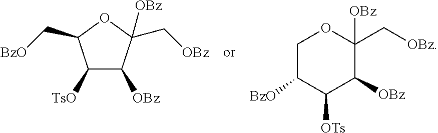

In another aspect, there is provided a precursor compound of formula (IIb) or (IIb′), or a pharmaceutically acceptable salt or ester thereof:

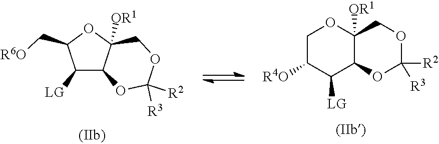

wherein: R¹ is lower alkyl; R² is H and R³ is phenyl, or R² and R³ are each independently lower alkyl; R⁴ and R⁶ are methoxymethyl (MOM), t-butyl (t-Bu), benzyl (Bn), trimethylsilyl (TMS), t-butyldimethylsilyl (TBDMS), or tetrahydropyranyl (THP); and LG is 4-methylphenylsulfonyl (—OTs), methanesulfonyl (˜OMs), trifluoromethansulfonyl (—OTf), 4-nitrophenylsulfonyl (˜ONs), phenylsulfonyl, —Br, —Cl, or —I.

In one embodiment, the precursor compound is

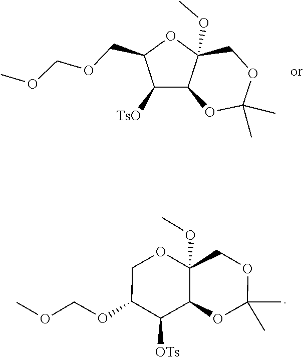

It should be understood that all acid, salt, base, and other ionic and non-ionic forms of compounds described herein are intended to be encompassed. For example, if a compound is shown as an acid herein, the salt forms of the compound are also encompassed. Likewise, if a compound is shown as a salt, the acid and/or basic forms are also encompassed.

Similarly, it should be understood that all tautomeric and isomeric forms of compounds described herein are intended to be encompassed. For example, sugars exist in equilibrium between their open-chain and various closed-chain forms (referred to as “ring-chain tautomerism”). The six-membered cyclic form is generally referred to as the “pyranose” form, while the five-membered cyclic form is generally referred to as the “furanose” form. It should be understood that if a compound is shown as a furanose form herein, the pyranose form is also encompassed, and vice-versa. Further, for sugars, closure of the ring creates a chiral center at C-1, resulting in two anomers or diastereomers (alpha and beta forms); all such forms are encompassed herein.

In certain embodiments of the compound of the disclosure, the compound of Formula (I) is suitable for use as an imaging agent. In particular, upon administration of the compound of Formula (I) to a subject, accumulation of the compound and metabolites of the compound in the one or more tissues can be measured using biomedical imaging techniques.

In some such embodiments, the compound is suitable for use for diagnostic imaging, e.g., nuclear imaging. In some such embodiments, the compound is suitable for use for PET, PET/CT and/or PET/MRI imaging. In some embodiments, the compound is suitable for use biomedical imaging, such as without limitation for diagnosis, monitoring, assessment, and/or treatment of fructolysis-associated disorders.

In certain embodiments, the compound is suitable for non-imaging tracer studies.

In certain embodiments of the compound of the disclosure, the compound is suitable for use as a therapeutic agent, e.g., to treat or prevent a fructolysis-associated disorder, such as a cancer.

In another aspect, there is provided a composition comprising a compound of Formula (I) and a carrier or excipient.

In certain embodiments, the composition is a pharmaceutical composition comprising a compound of Formula (I) in combination with one or more pharmaceutically acceptable carriers or excipients.

In certain embodiments, the composition is suitable for use as a nuclear imaging agent, e.g., for PET, PET/CT scanning and/or PET/MRI imaging.

In certain embodiments, the carrier is an aqueous solution. The carrier may be saline, water, phosphate-buffered saline (PBS), or dextrose 5% in water. Such compositions may be administered to a subject for biomedical applications such as imaging and/or treatment of a fructolysis-associated disorder.

In another aspect, there are provided methods of biomedical imaging, comprising administering a compound of the disclosure to a subject and imaging the compound and metabolites of the compound in the subject.

In certain embodiments, the biomedical imaging comprises positron emission tomography (PET). In some embodiments, the biomedical imaging comprises PET/CT. In some embodiments, the biomedical imaging comprises PET/MRI.

In certain embodiments, there is provided a method of imaging fructolysis in a subject, comprising administering a compound of the disclosure to the subject and imaging the compound and metabolites of the compound in the subject. In some such embodiments, the method further comprises imaging the compound in one or more tissue in the subject. In some embodiments, the imaging is PET, PET/CT or PET/MRI imaging. In certain embodiments, quantitative and/or qualitative functional information relating to fructose metabolism is obtained. Such methods may be used for example to diagnose, monitor and/or assess a fructolysis-associated disorder, e.g., a cancer, in a subject. In some embodiments, such methods may be used to monitor response to treatment in a subject undergoing treatment for one or more fructolysis-associated disorder.

In certain embodiments, there is provided a method of mapping neuroinflammation in a subject, comprising administering a compound of the disclosure to the subject and imaging the compound and metabolites of the compound in the subject, e.g., using PET or PET/CT or PET/MRI imaging.

In certain embodiments, there is provided a method of diagnosing a cancer in a subject, comprising administering an effective amount of a compound or composition of the disclosure to the subject and imaging one or more tissue in the subject using PET, the compound being capable of being retained within cancer tissue sufficient to emit positrons for detection by PET.

In certain embodiments, there is provided a method of monitoring a cancer in a subject, comprising administering an effective amount of a compound or composition of the disclosure to the subject and imaging one or more tissue in the subject using PET, the compound being capable of being retained within cancer tissue sufficient to emit positrons for detection by PET. In some such embodiments, the subject is undergoing medical treatment for the cancer. In some embodiments, such methods further comprise comparing the quantity or distribution of the compound present in the subject with a control quantity or distribution indicative of the effectiveness of the medical treatment.

In some embodiments, there is provided a method of treating or preventing a fructolysis-associated disorder in a subject in need thereof, comprising administering an effective amount of a compound or composition of the disclosure to the subject such that the fructolysis-associated disorder is treated or prevented.

In some embodiments of methods of the disclosure, the fructolysis-associated disorder is cardiac hypertrophy, myocardial infarction, a cardiovascular disease, a neurodegenerative disease (e.g., Alzheimer's disease), an ocular disease, a traumatic injury (e.g., traumatic brain injury), a stroke injury, a cancer, metabolic syndrome, obesity, diabetes, or inflammation (e.g., system inflammation, cardio-inflammation, neuroinflammation, etc.).

In some embodiments of methods of the disclosure, the cancer is a cancer of the brain, lung, breast, pancreas, kidney, colon, rectum, ovary, prostate, head, neck, thyroid, bladder, bone, endometrium, testes, esophagus, stomach, gastrointestinal system, glioblastoma, or neuroblastoma.

In another aspect, there is provided a method of treating cancer in a subject in need thereof, comprising administering an effective amount of the compound of Formula (I), wherein X is ²¹¹At, ¹³¹I, and ⁵⁷Br, or composition comprising the compound, to the subject, such that the compound can outcompete cancer cells for fructose in GLUT-mediated hexose uptake.

In certain embodiments of methods of the disclosure, the subject has, is suspected of having, or is at risk of a fructose-associated disorder. In certain embodiments of methods of the disclosure, the subject has, is suspected of having, or is at risk of cancer. In some embodiments, the subject is undergoing treatment for such a disorder or cancer. In some embodiments, the subject is a mammal, e.g., a human.

In certain embodiments of methods of the disclosure, the compound is 4-deoxy-4-fluoro-fructose ([¹⁸F]4-FDF).

From a yet further aspect, there is provided the compound or composition as described herein for use in imaging, such as biomedical nuclear imaging, such as PET or PET/CT or PET/MRI. In certain embodiments, the compound or composition is for use in imaging fructolysis and/or for diagnosis, monitoring, assessment, or treatment of a fructolysis-associated condition. In certain embodiments, the compound or composition is for use in imaging a cancer or tumor and/or for diagnosis, monitoring, assessment, or treatment of a cancer or tumor.

From a yet other aspect, there is provided the compound or composition as described and/or claimed herein for use as a PET imaging agent.

From a further aspect, there is provided a radiopharmaceutical for biomedical imaging of fructolysis comprising the compound or composition as described herein. In some embodiments, the compound is a compound of formula (I), or a pharmaceutically acceptable salt or ester thereof.

From another aspect, there is provided the compound or composition as described herein for use in treating or preventing a fructolysis-associated disorder in a subject in need thereof.

From yet another aspect, there is provided a kit comprising the compound, composition or imaging agent as described herein. Kits may further comprise a buffer or excipient, and/or instructions for use, e.g., in biomedical imaging.

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The terms “derivative” and “analog” are used interchangeably herein.

The term “lower alkyl” refers to a group having one to six carbon atoms in the chain which chain may be straight or branched. Non-limiting examples of suitable alkyl groups include methyl, ethyl, n-propyl, isopropyl, n-butyl, t-butyl, n-pentyl, and hexyl.

The term “biocompatible” as used herein means generating no significant undesirable response for the intended utility in the subject. In general, biocompatible materials are nontoxic for the intended utility. Thus, for human utility, biocompatible is preferably non-toxic and otherwise non-damaging to humans or human tissues.

As used herein, the term “carrier” means a suitable vehicle which is biocompatible and pharmaceutically acceptable, including for example and without limitation, liquid diluents which are suitable for administration.

As used herein, the term “effective amount” means any amount of a formulation of a compound of the disclosure, e.g., a radiopharmaceutical, useful for diagnostic imaging (e.g., of cancer cells) upon administration to a subject.

As used herein, the term “fructose-based” means a radiopharmaceutical which includes fructose, analogs or derivatives thereof.

As used herein, the term “pharmaceutically acceptable” means a substance which does not significantly interfere with the effectiveness of the compound of the disclosure for its intended use, and which has an acceptable toxicity or safety profile for the subject to which it is administered.

The term “subject,” as used herein, includes eukaryotes, such as mammals, including human or other mammalian subjects, e.g., humans, ovines, bovines, equines, porcines, canines, felines, non-human primates, mice, and rats. In certain embodiments, the subject is a human. In certain embodiments, the subject is a non-human mammal, such as, for example and without limitation, primates, livestock animals (e.g., sheep, cows, horses, goats, pigs), domestic companion animals (e.g., cats, dogs), laboratory test animals (e.g., mice, rats, guinea pigs, rabbits) or captive wild animals. The terms “subject” and “patient” are used interchangeably herein.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

It will be appreciated that a compound of the disclosure, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this disclosure, heteroatoms such as nitrogen may have hydrogen substituents and/or any permissible substituents of organic compounds described herein which satisfy the valencies of the heteroatoms. Furthermore, this disclosure is not intended to be limited in any manner by the permissible substituents of organic compounds.

The term “stable moiety,” as used herein, preferably refers to a moiety which possess stability sufficient to allow manufacture (including manufacture in situ), and which maintains its integrity for a sufficient period of time to be useful for the purposes detailed herein.

The term “stable radical,” as used herein, refers to a free radical that possesses stability sufficient to allow manufacture (including manufacture in situ), and which maintains its integrity for a sufficient period of time to be useful for the purposes detailed herein.

The present disclosure is directed to fructose-based radiopharmaceuticals, pharmaceutical compositions comprising same, methods for preparing same, and methods of using same for diagnostic medical imaging, e.g., of fructolysis-associated disorders.

Aspects of the present technology comprise compounds which are radiolabelled fructose derivatives, as described herein (e.g., compounds of Formula (I)). The compounds are useful clinically for medical imaging, including but not limited to PET, and can provide reliable, rapid, and/or quantitative clinical imaging to facilitate early diagnosis and detection of various fructolysis-associated disorders. Aspects of the present technology comprise compositions and pharmaceutical compositions including the compounds, methods of making such compounds, and uses thereof.

Radiopharmaceutical compounds of the disclosure may include any acceptable radionuclide including, but not limited to, fluorine-18, radioactive isotopes of iodine, bromine, chlorine, astatine and carbon, or others as will be apparent to those skilled in the art. In one embodiment, the radionuclide is fluorine (F), e.g., ¹⁸F. The incorporation of the selected radionuclide generally occurs in the final or next-to-final reactions of the overall synthesis, since the half-lives of particular radionuclides may be short; for example, the half-life of the [¹⁸F] radionuclide is about 110 minutes.

In one embodiment, the radiopharmaceutical of the disclosure is [¹⁸F]-4-deoxy-4-fluoro-fructose ([¹⁸F]4-FDF).

### Compositions

In certain embodiments, the compounds of the present disclosure can be present in a composition or a pharmaceutical composition.

Pharmaceutical compositions are generally formulated to be compatible with the intended method or route of administration; exemplary routes of administration include without limitation oral, by inhalation, or parenteral, e.g., intramuscular, intravenous, subcutaneous (e.g., injection or implant), intraperitoneal, intrathecal, or intraarticular. In some embodiments, the pharmaceutical composition is provided in a single-use container (e.g., a single-use vial, ampoule, syringe, or autoinjector, whereas a multi-use container (e.g., a multi-use vial) is provided in other embodiments. Compounds and compositions provided herein may be administered to a subject in any appropriate manner known in the art.

The term “pharmaceutically acceptable carrier” refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, compositions and pharmaceutical compositions of the disclosure are present in the form of micelles. For some micelles, a composition of the disclosure is mixed with a nonpolar radical, e.g., a radical containing a perfluorinated moiety. A composition or pharmaceutical composition may also include a surfactant. One non-limiting type of a perfluorinated radical is a TEMPO group with a fluorinated tail, e.g. a perfluorinated tail (described in Pozzi, Adv. Synth. Cat. 347:677 (2005), the contents of which are incorporated by reference). An exemplary perfluorinated radical is TEMPO attached to a C₆-C₂₀ (such as C₈-C₁₂) perfluoroalkyl group via an amide or sulfonamide group at the 4-position of TEMPO. Suitable surfactants include, without limitation, perfluorinated sulfonic carboxylic acids, particularly C₄-C₁₂ acids such as C₆, C₇, C₈, C₉, C₁₀, C₁₁ and C₁₂ acids. Exemplary surfactants include without limitation ammonium perfluorooctanoate (FC143), perfluorooctanesulfonic acid (PFOS) and perfluorononanoic acid (PFNA).

Compositions and pharmaceutical compositions of the disclosure may also include additional components such as stabilizers, preservatives, dispersants, and the like. Compositions and pharmaceutical compositions of the disclosure may also include additional components suitable for the intended purpose, e.g., suitable for imaging and/or administration to a subject, such as excipients, dyes, and the like.

A “pharmaceutically acceptable salt” of a compound means a salt of a compound that is pharmaceutically acceptable. Desirable are salts of a compound that retain or improve the biological effectiveness and properties of the free acids and bases of the parent compound as defined herein or that take advantage of an intrinsically basic, acidic or charged functionality on the molecule and that are not biologically or otherwise undesirable. Examples of pharmaceutically acceptable salts are also described, for example, in Berge et al., “Pharmaceutical Salts”, J. Pharm. Sci. 66, 1-19 (1977). Non-limiting examples of such salts include: (1) acid addition salts, formed on a basic or positively charged functionality, by the addition of inorganic acids such as hydrochloric acid, hydrobromic acid, hydroiodic acid, sulfuric acid, sulfamic acid, nitric acid, phosphoric acid, carbonate forming agents, and the like; or formed with organic acids such as acetic acid, propionic acid, lactic acid, oxalic, glycolic acid, pivalic acid, t-butylacetic acid, p-hydroxybutyric acid, valeric acid, hexanoic acid, cyclopentanepropionic acid, pyruvic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, cyclohexylaminosulfonic acid, benzenesulfonic acid, sulfanilic acid, 4-chlorobenzenesulfonic acid, 2-napthalenesulfonic acid, 4-toluenesulfonic acid, camphorsulfonic acid, 3-phenyl propionic acid, lauryl sulphonic acid, lauryl sulfuric acid, oleic acid, palmitic acid, stearic acid, lauric acid, embonic (pamoic) acid, palmoic acid, pantothenic acid, lactobionic acid, alginic acid, galactaric acid, galacturonic acid, gluconic acid, glucoheptonic acid, glutamic acid, naphthoic acid, hydroxynapthoic acid, salicylic acid, ascorbic acid, stearic acid, muconic acid, and the like; (2) base addition salts, formed when an acidic proton present in the parent compound either is replaced by a metal ion, including, an alkali metal ion (e.g., lithium, sodium, potassium), an alkaline earth ion (e.g., magnesium, calcium, barium), or other metal ions such as aluminum, zinc, iron and the like; or coordinates with an organic base such as ammonia, ethylamine, diethylamine, ethylenediamine, N,N′-dibenzylethylenediamine, ethanolamine, diethanolamine, triethanolamine, tromethamine, N-methylglucamine, piperazine, chloroprocain, procain, choline, lysine and the like.

Pharmaceutically acceptable salts may be synthesized from a parent compound that contains a basic or acidic moiety, by conventional chemical methods. Generally, such salts are prepared by reacting the free acid or base forms of compounds with a stoichiometric amount of the appropriate base or acid in water or in an organic solvent, or in a mixture of the two. Salts may be prepared in situ, during the final isolation or purification of a compound or by separately reacting a compound in its free acid or base form with the desired corresponding base or acid, and isolating the salt thus formed. The term “pharmaceutically acceptable salts” also include zwitterionic compounds containing a cationic group covalently bonded to an anionic group, as they are “internal salts”.

### Uses

Uses of compounds and compositions of the present technology are not limited and may include as agents for nuclear imaging, e.g. by PET, PET/CT or PET/MRI. Thus, in certain embodiments, compounds described herein are radiopharmaceuticals used for imaging, e.g., PET. It is to be understood that any suitable imaging technique may be used in conjunction with compounds and compositions described herein. It should be understood that the use of compounds and compositions described herein is not meant to be particularly limited; for example, compounds and compositions may be used for imaging a variety of tissues and conditions, depending on the compound's uptake/distribution after administration to a subject and other considerations that determine suitability for a particular use.

In some embodiments, compounds and compositions of the present technology are useful for imaging fructolysis. Fructolysis occurs during the onset and progression of a variety of diseases, where inflammation-induced energy crises activate a fructolytic state in the affected tissues. The ability to map fructose metabolism, including detection and monitoring thereof, may assist with diagnosis, monitoring, and/or treatment of a wide range of fructolysis-associated disorders.

Examples of fructolysis-associated disorders include, without limitation: cardiac hypertrophy, myocardial infarction, a cardiovascular disease, a neurodegenerative disease (e.g., Alzheimer's disease), an ocular disease, a traumatic injury (e.g., traumatic brain injury), a stroke injury, a cancer, metabolic syndrome, obesity, diabetes, or inflammation (e.g., system inflammation, cardio-inflammation, neuroinflammation, etc.).

Examples of cancers include, without limitation: cancers of the brain, lung, breast, pancreas, kidney, colon, rectum, ovary, prostate, head, neck, thyroid, bladder, bone, endometrium, testes, esophagus, stomach, gastrointestinal system, blood, lymphatics, leukemia, glioblastoma, or neuroblastoma. In some embodiments, a cancer is a solid tumor. In some embodiments, a cancer is a GLUT5 and/or ketohexokinase-expressing tumor.

In some embodiments, compounds and compositions of the present technology are useful for treating or preventing a fructolysis-associated disorder.

In some embodiments, there are provided methods of biomedical imaging, comprising administering an effective amount of a compound of the disclosure or a pharmaceutically acceptable salt or ester thereof, or a composition thereof, to the subject, and imaging one or more tissue in the subject, e.g., using nuclear imaging methods to measure accumulation of compounds of Formula I and the metabolites of the compound in the tissue. In some such embodiments, evidence of fructolysis is imaged in the subject. In some embodiments, the nuclear imaging method comprises PET/CT. In some embodiments, the nuclear imaging method comprises PET/MRI.

In some embodiments, there are provided methods of diagnosing and/or monitoring a fructolysis-associated disorder in a subject, comprising administering an effective amount of a compound of the disclosure or a pharmaceutically acceptable salt or ester thereof, or a composition thereof, to the subject, and imaging one or more tissue in the subject, e.g., using positron emission tomography (PET) or PET/CT or PET/MRI to measure accumulation of compounds of Formula I and the metabolites of the compound in the tissue.

In some embodiments, there are provided methods of diagnosing and/or monitoring cancer in a subject, comprising administering an effective amount of a compound of the disclosure or a pharmaceutically acceptable salt or ester thereof, or a composition thereof, to the subject, and imaging one or more tissue in the subject, e.g., using positron emission tomography (PET) or PET/CT or PET/MRI to measure accumulation of compounds of Formula I and the metabolites of the compound in the tissue. In some such embodiments, the subject is undergoing medical treatment for cancer, and the quantity and/or distribution of the compounds of Formula I and the metabolites of the compound is compared with a control quantity and/or distribution indicative of the effectiveness of the medical treatment.

In some embodiments, there are provided methods of monitoring cancer progression in response to anti-cancer treatment, e.g., with one or more chemotherapeutic agent, comprising administering an effective amount of a compound of the disclosure or a pharmaceutically acceptable salt or ester thereof, or a composition thereof, to a subject who in undergoing the anti-cancer treatment and imaging the cancer in the subject, e.g., using positron emission tomography (PET) or PET/CT or PET/MRI.

In some embodiments, there are provided methods of treating or preventing a fructolysis-associated disorder in a subject, comprising administering an effective amount of a compound of the disclosure or a pharmaceutically acceptable salt or ester thereof, or a composition thereof, to the subject, such that the fructolysis-associated disorder is treated or prevented in the subject. In some such embodiments, the fructolysis-associated is a cancer, e.g., a cancer of the brain, lung, breast, pancreas, kidney, colon, rectum, ovary, prostate, head, neck, thyroid, bladder, bone, endometrium, testes, esophagus, stomach, gastrointestinal system, blood, lymphatics, leukemia, glioblastoma, or neuroblastoma. In some such embodiments, the fructolysis-associated is Alzheimer's disease, cardiovascular disease, an inflammatory condition, or metabolic syndrome. In some such embodiments, the subject is a human. In some such embodiments, the subject is a non-human mammal, e.g., a non-human primate, canine, feline, etc.

In some embodiments, there are provided methods of treating cancer in a subject, comprising administering an effective amount of a compound of Formula (I) wherein X is ²¹¹At, ¹³¹I, and ⁵⁷Br, or a pharmaceutically acceptable salt or ester thereof, or a composition thereof, to the subject, and imaging one or more tissue in the subject. In some such embodiments, the compound of the disclosure outcompetes cancer cells for fructose in GLUT-mediated hexose uptake.

In some embodiments, the compound of the disclosure or the pharmaceutically acceptable salt or ester thereof, or the composition thereof is useful clinically as a non-imaging tracer.

In certain embodiments of imaging methods of the disclosure, quantitative and/or qualitative functional information relating to fructose metabolism is obtained. In some embodiments, reliable, rapid, and/or quantitative clinical nuclear imaging for mapping and measuring fructose metabolism is provided.

In certain embodiments of methods provided herein, the subject has, is suspected of having, or is at risk of a fructolysis-associated disorder. A subject may have, be suspected of having, or be at risk of cancer, Alzheimer's disease, cardiovascular disease, an inflammatory condition, metabolic syndrome, etc. A subject may be undergoing medical treatment for a fructolysis-associated disorder, e.g., a cancer.

### Kits

There are also provided herein kits comprising a compound or composition as described herein. Kits are generally in the form of a physical structure housing various components and may be used, for example, in practicing the methods provided herein. For example, a kit may include one or more compound or composition disclosed herein (provided in, e.g., a sterile container). The compound or composition can be provided in a form that is ready for use or in a form requiring, for example, reconstitution or dilution (e.g., a powder). When the compounds or compositions are in a form that needs to be reconstituted or diluted by a user, the kit may also include diluents (e.g., sterile water), buffers, pharmaceutically acceptable excipients, and the like, packaged with or separately from the compounds or compositions. Each component of the kit may be enclosed within an individual container, and all of the various containers may be within a single package. A kit of the present invention may be designed for conditions necessary to properly maintain the components housed therein (e.g., refrigeration or freezing).

In one embodiment, the kit comprises a sample of the protected precursor. In one embodiment, the precursor is a compound of Formula (IIa) or (IIa′) or a compound of Formula (IIb) or (IIb′). In a preferred embodiment, the precursor is Compound 5.

The kit in accordance with the present disclosure further includes reagents required for deprotecting the protected precursor to provide the deprotected radionuclide labeled fructose derivative. In one embodiment, the deprotected radiolabeled fructose derivative is [¹⁸F]4-FDF (Compound 7).

A further embodiment of the kit in accordance with the present disclosure further includes the physical equipment suitable for carrying out the deprotection and subsequent purification steps, including but not limited to vials, tubing, solid phase extraction columns, and instructions for carrying out the deprotection and purification steps.

In one embodiment, the kit may be adapted for use with commercial synthesis modules (e.g. Trasis, G E, Synthra) and may include a software component that works with proprietary control software to provide a time list of commands to allow automated production of the final radiolabeled fructose derivative.

The kit may also contain a label or packaging insert including identifying information for the components therein and instructions for their use. Labels or inserts can include manufacturer information such as lot numbers and expiration dates. The label or packaging insert may be, e.g., integrated into the physical structure housing the components, contained separately within the physical structure, or affixed to a component of the kit (e.g., an ampoule, tube or vial).

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Synthetic Procedures

### Example 1. Synthesis of [U¹³C]-1-fluoro-1-deoxyfructose ([U¹³C]1-FDF)

Synthesis of [UL-¹³C₆]-1-F-D-fructose adopted the following synthetic pathway adapted from the literature. Protection of D-fructose with acetone in the presence of H₂SO₄ resulted in the formation of 2,3-di-O-isopropylidene-4,5-di-O-isopropylidene-β-D-fructopyranose. This procedure was adopted from the literature (Sandner, A. et al., 2021). Corresponding protected material was converted to 1-OTf-2,3-di-O-isopropylidene-4,5-di-O-isopropylidene-β-D-fructopyranose; followed by TBAF-mediated nucleophilic fluorination, as described (Pérez-Tomas, R. et al., 2020). Final deprotection was carried out by Amberlyst 15-mediated acidolysis, as described (Wang, Z-H et al., 2021). The spectral characterization of the intermediates in the original literature is incomplete (Sandner, A. et al., 2021; Pérez-Tomás, R. et al., 2020), and therefore detailed spectral characterization was carried out. The synthesis is depicted in Scheme 1.

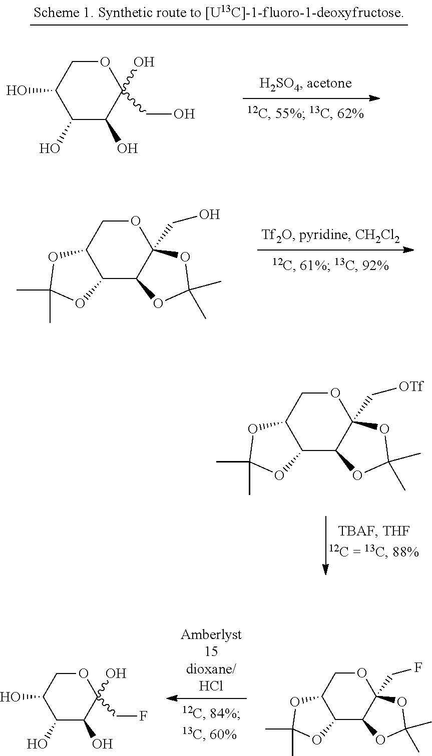

[UL-¹³C₆]-2,3-Di-O-isopropylidene-4,5-di-O-isopropylidene-β-D-fructopyranose. A stirred solution of H₂SO₄ (440 μL, 8.3 mmol) in dry acetone (8.8 mL) was cooled to 0° C., followed by the addition of [UL-¹³C₆]-D-fructose (465 mg, 2.5 mmol). The cooling bath was removed, and the mixture was stirred for 90 minutes at rt. The mixture was cooled to 0° C. and the reaction was quenched by a slow addition of a solution prepared by dissolving 1.38 g NaOH in 6.3 mL of water. Acetone was evaporated, the aqueous solution was diluted with water (10 mL) and was extracted with CH₂Cl₂ (3×10 mL). Combined organic extract was dried and was concentrated to leave colorless solid. The material was dissolved in Et₂O (3 mL), was diluted with n-pentane (5 mL) and was set aside for 3 h at −10° C. The crystals were filtered off, were washed with small amount of n-pentane, and were dried to afford 359 mg (54%) of the desired product. The mother liquor was concentrated, and the residue was subjected to FCC on 30 g SiO₂, hexanes/EtOAc (2:1). Evaporation of the eluate afforded second crop of the desired product as colorless solid, 55 mg (8%). [UL-¹³C₆]-2,3-Di-O-isopropylidene-4,5-di-O-isopropylidene-β-D-fructopyranose, colorless solid (414 mg, 62%). ¹H NMR (CDCl₃) δ 4.87-3.86 (m, 5H); 3.78-3.38 (m, 2H); 2.13 (s, D₂O exch., 1H); 1.54 (s, 3H); 1.47 (s, 3H); 1.39 (d, J=0.5 Hz, 3H); 1.34 (d, J=0.5 Hz, 3H). ¹H{¹³C} NMR (CDCl₃) δ 4.60 (dd, J=8.0, 3.0 Hz, 1H); 4.33 (d, J=3.0 Hz, 1H); 4.23 (d, J=8.0 Hz, 1H); 3.91 (dd, J=13.0, 2.0 Hz, 1H); 3.76 (d, J=13.0 Hz, 1H); 3.67 (dd, J=15.5, 11.5 Hz, 1H); 2.13 (s, D₂O exch., 1H); 1.54 (s, 3H); 1.47 (s, 3H); 1.39 (s, 3H); 1.34 (s, 3H). ¹³C NMR (CDCl₃) δ 103.7-102.4 (m); 71.6-69.3 (m); 65.8-65.1 (m); 61.5-60.9 (m); 26.4; 25.7; 25.3; 23.9. HRMS (ESI) m/z found 289.1350 [M+Na]⁺ (289.1359 calcd for ¹²C₆¹³C₆H₂₀O₆Na).

[UL-¹³C₆]-1-OTf-2,3-di-1-isopropylidene-4,5-di-O-isopropylidene-β-D-fructopyranose. A solution of [UL-¹³C]-2,3-di-O-isopropylidene-4,5-di-O-isopropylidene-β-D-fructopyranose (120 mg, 0.45 mmol) in dry CH₂Cl₂ (8 mL) and dry pyridine (200 μL) was cooled to −20° C., followed by the addition of Tf₂O (90 μL, 0.54 mmol). The mixture was stirred for 90 minutes at −20° C., the reaction was quenched by the addition of saturated NaHCO₃ solution and CH₂Cl₂ (10 mL each). The organic phase was separated, and the aqueous phase was extracted with CH₂Cl₂ (2×15 mL). Combined organic extract was dried, and the residue was subjected to FCC on 25 g SiO₂, petroleum ether/EtOAc (7:3). Evaporation of the eluate afforded colorless oil, [UL-¹³C]-1-OTf-2,3-di-O-isopropylidene-4,5-di-O-isopropylidene-β-D-fructopyranose (166 mg, 92%). ¹H NMR (CDCl₃) δ 4.89-3.97 (m, 6H); 3.72-3.52 (m, 1H); 1.56 (s, 3H); 1.47 (s, 3H); 1.40 (d, J=0.5 Hz, 3H); 1.35 (d, J=0.5 Hz, 3H). ¹H{¹³C} NMR (CDCl₃) δ 4.64 (dd, J=8.0, 3.0 Hz, 1H); 4.52 (d, J=10.5 Hz, 1H); 4.40 (d, J=10.5 Hz, 1H); 4.31 (d, J=3.0 Hz, 1H); 4.25 (d, J=8.0 Hz, 1H); 3.93 (dd, J=13.0, 2.0 Hz, 1H); 3.78 (d, J=13.0 Hz, 1H); 1.56 (s, 3H); 1.47 (s, 3H); 1.40 (s, 3H); 1.35 (s, 3H). ¹³C NMR (CDCl₃) δ 118.6 (q, J=317.5 Hz), 100.4-99.2 (m); 74.4-73.6 (m); 71.0-68.9 (m); 62.1-61.2 (m); 26.5, 25.7, 25.0, 23.9. ¹⁹F NMR (CDCl₃) δ −74.4 (s).

[UL-¹³C₆]-1-F-2,3-di-1-isopropylidene-4,5-di-O-isopropylidene-β-D-fructopyranose. [UL-¹³C₆]-1-OTf-2,3-di-O-isopropylidene-4,5-di-O-isopropylidene-β-D-fructopyranose (166 mg, 0.42 mmol) was dissolved in 1 M TBAF solution in THF (1.9 mL, 1.9 mmol). The mixture was stirred for 18 h at rt, the solvent was evaporated, and the residue was subjected to FCC on 25 g SiO₂, hexanes/EtOAc (9:1). Evaporation of the eluate afforded colorless oil, [UL-¹³C]-1-F-2,3-di-O-isopropylidene-4,5-di-O-isopropylidene-β-D-fructopyranose (99 mg, 88%). ¹H NMR (CDCl₃) 4.90-3.97 (m, 6H); 3.71-3.64 (m, 0.5H); 3.54-3.45 (m, 0.5H); 1.55 (s, 3H); 1.46 (s, 3H); 1.40 (s, 3H); 1.35 (d, J=0.5 Hz, 3H). ¹H{¹³C} NMR (CDCl₃) δ 4.62 (dd, J=8.0, 3.0 Hz, 1H); 4.46 (m, 1H); 4.39 (dd, J=3.0, 1.5 Hz, 1H); 4.30 (dd, J=4.5, 1.5 Hz, 1H); 4.24 (m, 1H); 3.91 (dd, J=13.0, 2.0 Hz, 1H); 3.75 (d, J=13.0, 1H); 1.55 (s, 3H); 1.46 (s, 3H); 1.40 (s, 3H); 1.34 (s, 3H). ¹H{¹⁹F} NMR (CDCl₃) δ 4.88-3.46 (m, 7H); 1.55 (s, 3H); 1.46 (s, 3H); 1.40 (d, J=0.5 Hz, 3H); 1.35 (d, J=0.5 Hz, 3H). ¹³C NMR (CDCl₃) δ 101.9-100.4 (m); 83.5-80.3 (m); 71.3-68.8 (m); 61.6-61.4 (m); 26.5; 25.8; 25.1 (d, J=3.0 Hz); 24.0. ¹⁹F NMR (CDCl₃) δ −229.6 to −230.6 (m). ¹⁹F{¹H} NMR (CDCl₃) 5-229.8 to −230.5 (m). HRMS (ESI) m/z found 291.1323 [M+Na]⁺ (291.1316 calcd for ¹²C₆¹³C₆H₁₉FO₅Na).

[UL-¹³C₆]-1-F-D-fructose. Amberlyst 15 (100 mg) was added to a solution of 1-F-2,3-di-O-isopropylidene-4,5-di-O-isopropylidene-β-D-fructopyranose (212 mg, 0.79 mmol) in dioxane/2 M HCl (1.5 mL each). The mixture was stirred for 72 h at rt. The resin was filtered off (Pasteur pipette with a cotton plug), the filtrate was cooled to 0° C. and the pH was adjusted to ˜6-7 (20% NaOH solution). The solvent was evaporated, and the residue was subjected to FCC on 25 g SiO₂, CH₂Cl₂/MeOH (9:1), later replaced with CH₂Cl₂/MeOH (4:1). Evaporation of the eluate afforded colorless oil, this material was dissolved in small amount of water (˜5 mL), was transferred to a Falcon tube, was frozen and was lyophilized to afford colorless sticky solid, [UL-¹³C₆]-4-iodo-D-fructose (89 mg, 60%). ¹H NMR (D₂O) δ 4.86-3.42 (m, 7H). ¹H{¹³C} NMR (D₂O) δ 4.61-4.24 (m, 2H); 4.15-3.63 (m, 51H). ¹H{¹⁹F} NMR (D₂O) δ 4.75-3.39 (m, 7H). ¹³C NMR (D₂O) δ 100.5-98.8 (m, minor); 97.6-96.0 (m, major); 84.7-80.2 (m, major+minor); 76.1-73.4 (m, major+minor); 69.9-68.3 (m, major); 67.8-66.4 (m, minor); 64.0-60.6 (m, major+minor). ¹⁹F NMR (D₂O) δ −228.4 to −229.4 (m, minor); −230.2 to −231.2 (m, minor); −231.6 to −232.6 (m, major). ¹⁹F{¹H} NMR (D₂O) δ −228.5 to −229.2 (m, minor); −230.4 to −231.1 (m, minor); −231.7 to −232.4 (m, major). HRMS (ESI) m/z found 211.0688 [M+Na]⁺ (211.0690 calcd for ¹³C₆H₁₁FO₅Na).

### Example 2. Synthesis of [U¹³C]-6-fluoro-6-deoxyfructose ([U¹³C]6-FDF)

Preparation of both anomers of [UL-¹³C₆]-1,3,4-tri-OAc-methyl-D-fructofuranose was adopted from the previously published well documented protocol used for the preparation of “natural” carbon isotope containing 6-F-D-fructose (Sasa, K. et al., 2018). “Natural” 6-F-D-fructose was also prepared for comparison, using the procedures described in the literature (Sasa, K. et al., 2018). Spectral characterization of all intermediates matched that described in the literature (Johnson, R. J. et al., 2020).

Both anomers of [UL-¹³C₆]-1,3,4-tri-OAc-methyl-D-fructofuranose were converted to [UL-¹³C₆]-6-OTf-1,3,4-tri-OAc-methyl-D-fructofuranose, followed by CsF-mediated nucleophilic replacement to give [UL-¹³C₆]-6-F-1,3,4-tri-OAc-methyl-D-fructofuranose. Global deprotection was carried out in two steps: MeONa/MeOH was used to remove acetyl protecting groups, while treatment with diluted HCl resulted in removal of the acetal protecting group.

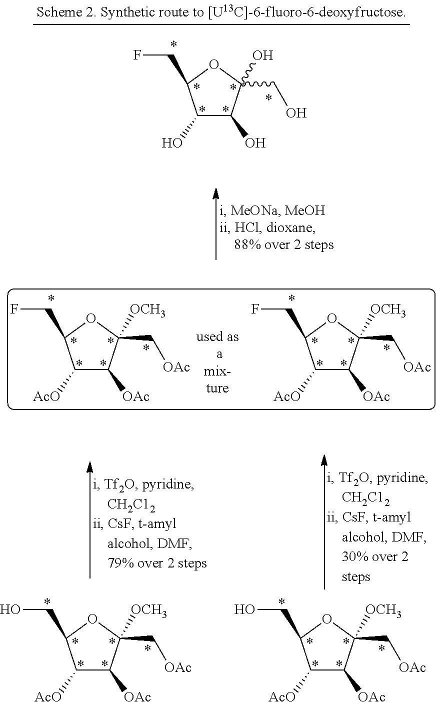

[UL-¹³C₆]-1,3,4-tri-OAc-methyl-D-fructofuranoside (α and β anomer). Amberlyst 15 (50 mg) was added to a stirred solution of [UL-¹³C₆]-D-fructose (465 mg, 2.5 mmol) in MeOH (25 mL). The mixture was stirred for 18 h at rt, the resin was filtered off, the filter was washed with MeOH and the filtrate was concentrated. The residue was subjected to FCC on 45 g SiO₂, CH₂Cl₂/MeOH (4:1). Evaporation of the eluate afforded colorless oil, [UL-¹³C₆]-methyl-D-fructofuranoside (224 mg, 45%, mixture of α- and β-anomer).

[UL-¹³C₆]-methyl-D-fructofuranoside (224 mg, 1.12 mmol, mixture of α- and β-anomer) was dissolved in dry pyridine (2.2 mL) and the mixture was cooled to 0° C. TMDMS-Cl (270 mg, 1.6 mmol) was added, the cooling bath was removed, and the mixture was stirred for 18 h at rt. Toluene (120 mL) was added, the liquids were evaporated, and the residue was subjected to FCC on 50 g SiO₂, CH₂Cl₂/MeOH (9:1). Evaporation of the eluate afforded colorless oil, [UL-¹³C₆]-6-OTBDMS-methyl-D-fructofuranoside (182 mg, 52%, mixture of α- and β-anomer).

[UL-¹³C₆]-6-OTBDMS-methyl-D-fructofuranoside (182 mg, 0.58 mmol, mixture of α- and β-anomer) was dissolved in dry pyridine (2 mL) and the mixture was cooled to 0° C. Ac₂O (1 ml) was added, the cooling bath was removed, and the mixture was stirred for 18 h at rt. The mixture was co-evaporated with toluene (2×100 mL) to leave crude [UL-¹³C₆]-6-OTBDMS-1,3,4-tri-OAc-methyl-D-fructofuranoside (mixture of α- and β-anomer). This material was dissolved in CH₂Cl₂ (5 mL), water (150 μL) and TFA (1 mL) were added, and the mixture was stirred for 30 min at rt. The mixture was diluted with CH₂Cl₂ (50 mL), was dried (Na₂SO₄), and was concentrated. The residue was subjected to FCC on 40 g SiO₂, hexanes/EtOAc (1:1). Two fractions were obtained, less polar fraction, colorless oil, [UL-¹³C₆]-1,3,4-tri-OAc-methyl-α-D-fructofuranoside (63 mg, 33%, 8% overall); more polar fraction, colorless oil, [UL-¹³C₆]-1,3,4-tri-OAc-methyl-β-D-fructofuranoside (44 mg, 23%, 5% overall).

[UL-¹³C₆]-1,3,4-tri-OAc-methyl-α-D-fructofuranoside (63 mg, 33%, 8% overall). ¹H NMR (CDCl₃) δ 5.59-4.67 (m, 2H); 4.38-3.51 (m, 5H); 3.32 (d, J=4.0 Hz, 3H); 2.10 (s, 3H); 2.08 (s, 3H); 2.06 (s, 3H); 2.00 (brs, D₂O exch., 1H). ¹H NMR (CDCl₃, ¹³C-decoupled) 5.32 (m, 1H); 4.99 (m, 1H); 4.45 (d, J=12.5 Hz, 1H); 4.10 (d, J=12.5 Hz, 1H); 4.02 (dd, J=8.5, 4.0 Hz, 1H); 3.88 (dd, J=12.5, 3.5 Hz, 1H); 3.78 (dd, J=12.0, 4.0 Hz, 1H); 3.32 (s, 3H); 2.10 (s, 3H); 2.08 (s, 3H); 2.06 (s, 3H); 2.00 (br s, D₂O exch., 1H). ¹³C NMR (CDCl₃) δ 170.6 (q, J=2.5 Hz); 170.2 (t, J=2.5 Hz); 169.0 (s); 107.3-105.8 (m); 107.3-105.8 (m); 83.5-82.4 (m); 80.5-79.3 (m); 78.4-77.3 (m); 62.0 (d, J=41 Hz); 58.5 (dd, J=25.5, 3.0 Hz); 48.7 (m); 20.8 (s); 20.7 (2×s). HRMS (ESI) m/z found 349.1214 [M+Na]⁺ (349.1206 calcd for ¹²C₇¹³C₆H₂₀O₉Na).

[UL-¹³C₆]-1,3,4-tri-OAc-methyl-β-D-fructofuranoside (44 mg, 23%, 5% overall). ¹H NMR (CDCl₃) δ 5.84-4.98 (m, 2H); 4.52-3.45 (m, 5H); 3.36 (d, J=3.5 Hz, 3H); 2.65 (br s, D₂O exch., 1H); 2.10 (s, 3H); 2.08 (s, 6H). ¹H NMR (CDCl₃, ¹³C-decoupled) 5.50 (d, J=7.5 Hz, 1H); 5.33 (m, 1H); 4.24 (d, J=11.5 Hz, 1H); 4.14 (d, J=12.0 Hz, 1H); 4.00 (dd, J=11.0, 6.0 Hz, 1H); 3.81 (m, 1H); 3.69 (dd, J=12.0, 5.5 Hz, 1H); 3.36 (s, 3H); 2.64 (br s, D₂O exch., 1H); 2.10 (s, 3H); 2.08 (s, 6H). ¹³C NMR (CDCl₃) δ 171.0 (s); 170.1 (t, J=3.0 Hz); 169.9 (s); 103.4-101.4 (m); 81.5-79.9 (m); 77.4-75.4 (m); 71.0-67.3 (m); 63.4 (d, J=163.5 Hz); 62.2 (dd, J=216.5, 1.0 Hz); 49.7 (s); 20.8 (s); 20.7 (2×s). HRMS (ESI) m/z found 349.1198 [M+Na]⁺ (349.1206 calcd for ¹²C₇¹³C₆H₂₀ONa).

[UL-¹³C₆]-6-F-1,3,4-tri-OAc-methyl-D-fructofuranoside (α and β anomer). A solution of [UL-¹³C₆]-1,3,4-tri-OAc-methyl-α-D-fructofuranoside (177 mg, 0.54 mmol) in dry CH₂Cl₂ (5 mL) and dry pyridine (130 μL) was cooled to −20° C., followed by the addition of Tf₂O (110 μL, 0.65 mmol). The mixture was stirred for 30 minutes at −20° C., the reaction was quenched by the addition of water and CH₂Cl₂ (10 mL each). The organic phase was separated, the aqueous phase was extracted with CH₂Cl₂ (2×10 mL). Combined organic extract was consecutively washed with 5% aqueous H₂SO₄ solution and water (10 mL each), was dried, and was concentrated to leave an orange oil, crude [UL-¹³C₆]-6-OTf-1,3,4-tri-OAc-methyl-α-D-fructofuranoside used for the next reaction immediately. [UL-¹³C₆]-6-OTf-1,3,4-tri-OAc-methyl-α-D-fructofuranoside obtained in the previous step was dissolved in t-amyl alcohol (1.7 mL), CsF (247 mg, 1.63 mmol) was added, and the mixture was stirred for 20 minutes at 90° C. The reaction was quenched by the addition of water and CH₂Cl₂ (10 mL each). The organic phase was separated, the aqueous phase was extracted with CH₂Cl₂ (2×15 mL). Combined organic extract was washed with brine (2×15 mL), was dried and was subjected to FCC on 30 g SiO₂, petroleum ether/EtOAc (7:3). Evaporation of the eluate afforded colorless oil, [UL-¹³C₆]-6-F-1,3,4-tri-OAc-methyl-α-D-fructofuranoside (140 mg, 79% over 2 steps). ¹H NMR (CDCl₃) δ 5.56-3.88 (m, 7H); 3.32 (d, J=4.0 Hz, 3H); 2.11 (s, 3H); 2.08 (s, 3H); 2.05 (s, 3H). ¹H{¹³C} NMR (CDCl₃) δ 5.30 (m, 1H); 4.94 (m, 1H); 4.74 (dd, J=10.5, 2.5 Hz, 0.5H); 4.68 (dd, J=11.0, 5.5 Hz, 0.5H); 4.59 (dd, J=10.5, 2.5 Hz, 0.5H); 4.53 (dd, J=11.0, 5.5 Hz, 0.5H); 4.46 (d, J=12.0, 1H); 4.15 (d, J=12.0, 1H); 4.15 (m, 0.5H); 4.07 (m, 0.5H); 3.32 (s, 3H); 2.11 (s, 3H); 2.08 (s, 3H); 2.05 (s, 3H). ¹H{¹⁹F} NMR (CDCl₃) δ 5.56-3.85 (m, 7H); 3.32 (d, J=4.0 Hz, 3H); 2.11 (s, 3H); 2.08 (s, 3H); 2.05 (s, 3H). ¹³C NMR (CDCl₃) δ 170.3; 170.1 (t, J=2.0 Hz); 169.0 (t, J=2.5 Hz); 107.7-106.2 (m); 83.1-79.0 (m); 77.8-76.6 (m); 58.4-57.6 (m); 48.7, 20.7 (3×C). ¹⁹F NMR (CDCl₃) δ −229.2 to −230.5 (m). ¹⁹F{¹H} NMR (CDCl₃) δ −229.5 to −230.3 (m). HRMS (ESI) m/z found 351.1162 [M+Na]⁺ (351.1163 calcd for ¹²C₇¹³C₆H₁₉FO₈Na).

A solution of [UL-¹³C₆]-1,3,4-tri-OAc-methyl-β-D-fructofuranoside (73 mg, 0.22 mmol) in dry CH₂Cl₂ (2 mL) and dry pyridine (22 μL) was cooled to −20° C., followed by the addition of Tf₂O (45 μL, 0.27 mmol). The mixture was stirred for 30 minutes at −20° C., the reaction was quenched by the addition of water and CH₂Cl₂ (7 mL each). The organic phase was separated, the aqueous phase was extracted with CH₂Cl₂ (2×7 mL). Combined organic extract was consecutively washed with 5% aqueous H₂SO₄ solution and water (7 mL each), was dried, and was concentrated to leave an orange oil, crude [UL-¹³C₆]-6-OTf-1,3,4-tri-OAc-methyl-β-D-fructofuranoside used for the next reaction immediately. [UL-¹³C₆]-6-OTf-1,3,4-tri-OAc-methyl-β-D-fructofuranoside obtained in the previous step was dissolved in t-amyl alcohol (700 μL), CsF (102 mg, 0.67 mmol) was added, and the mixture was stirred for 20 minutes at 90° C. The reaction was quenched by the addition of water and CH₂Cl₂ (7 mL each). The organic phase was separated, the aqueous phase was extracted with CH₂Cl₂ (2×7 mL). Combined organic extract was washed with brine (2×15 mL), was dried and was subjected to FCC on 10 g SiO₂, petroleum ether/EtOAc (7:3). Evaporation of the eluate afforded colorless oil, [UL-¹³C₆]-6-F-1,3,4-tri-OAc-methyl-β-D-fructofuranoside (140 mg, 79% over 2 steps). ¹H NMR (CDCl₃) δ 5.84-3.88 (m, 7H); 3.38 (d, J=4.0 Hz, 3H); 2.12 (s, 3H); 2.10 (s, 3H); 2.09 (s, 3H). ¹H{¹³C} NMR (CDCl₃) δ 5.52 (d, J=7.5 Hz, 1H); 5.40 (m, 1H); 4.72 (dd, J=11.5, 3.5 Hz, 0.5H); 4.57 (m, 1H); 4.41 (dd, J=10.5, 6.0 Hz, 0.5H); 4.26 (d, J=12.0, 1H); 4.21 (m, 0.5H); 4.16 (d, J=12.0, 1H); 4.12 (m, 0.5H); 3.38 (s, 3H); 2.12 (s, 3H); 2.10 (s, 3H); 2.09 (s, 3H). ¹H{¹⁹F} NMR (CDCl₃) b 5.84-3.89 (m, 7H); 3.38 (d, J=4.0 Hz, 3H); 2.12 (s, 3H); 2.10 (s, 3H); 2.09 (s, 3H). ¹³C NMR (CDCl₃) δ 170.3; 170.1 (t, J=3.0 Hz); 170.0; 103.5-102.0 (m); 84.0-83.4 (m); 81.7-81.1 (m); 79.6-78.2 (m); 76.8-74.0; 62.4-61.7 (m); 49.7, 20.7 (3×C). ¹⁹F NMR (CDCl₃) δ −227.6 to −228.7 (m). ¹⁹F{¹H} NMR (CDCl₃) δ −227.8 to −228.5 (m). HRMS (ESI) m/z found 351.1147 [M+Na]⁺ (351.1163 calcd for ¹²C₇¹³C₆H₁₉FO₈Na).

[UL-¹³C₆]-6-F-D-fructose. A mixture of [UL-¹³C₆]-6-F-1,3,4-tri-OAc-methyl-α-D-fructofuranoside and [UL-¹³C₆]-6-F-1,3,4-tri-OAc-methyl-β-D-fructofuranoside (160 mg, 0.49 mmol) was dissolved in dry MeOH (4.5 mL) followed by the addition of a solution of MeONa in MeOH (1.5 M, 110 μL, 0.16 mmol). The mixture was stirred for 10 minutes at rt, the reaction was quenched with 1 M HCl (5 drops, pH˜6) and the solvent was evaporated. The residue was dissolved in dioxane (1.2 mL), 1 M HCl (800 μL) was added, and the mixture was stirred for 18 h at rt. The mixture was cooled to 0° C. and the pH was adjusted to ˜6-7 (20% NaOH solution). The solvent was evaporated, and the residue was subjected to FCC on 12 g SiO₂, CH₂Cl₂/MeOH (9:1). Evaporation of the eluate afforded colorless oil, this material was dissolved in small amount of water (˜5 mL), was transferred to a Falcon tube, was frozen and was lyophilized to afford colorless sticky solid, [UL-¹³C₆]-6-F-D-fructose (81 mg, 88%). ¹H NMR (D₂O) δ 4.94-4.72 (m, 1H); 4.47-3.64 (m, 5H); 3.40-3.29 (m, 1H). ¹H{¹³C} NMR (D₂O) δ 4.73-4.44 (m, 2H); 4.24-3.89 (m, 3H); 3.64-3.51 (m, 2H). ¹H{¹⁹F}NMR (D₂O) δ 4.90-4.77 (m, 1H); 4.45-3.70 (m, 5H); 3.40-3.29 (m, 1H). ¹³C NMR (D₂O) δ 105.3-104.0 (m, minor); 102.3-101.0 (m, major); 84.3-83.2 (m, major+minor); 82.1-81.0 (m, major+minor); 80.2-78.0 (m, major+minor); 75.5-74.3 (m, major+minor); 73.5-72.3 (m, major+minor); 62.8-62.0 (m, major+minor). ¹⁹F NMR (D₂O) δ −228.5 to −229.6 (m, major+minor). ¹⁹F{¹H} NMR (D₂O) δ −228.7 to −229.4 (m, major); −228.9 to −229.5 (m, minor). HRMS (ESI) m/z found 211.0686 [M+Na]⁺ (211.0690 calcd for ¹³C₆H₁₁FO₅Na).

### Example 3. Synthesis of [U¹³C]-4-fluoro-4-deoxyfructose ([U¹³C]4-FDF)

The preparation of [UL-¹³C₆]-4-F-D-fructose from [UL-¹³C₆]-6-MOM-methyl-1,3-di-O-isopropylidene-α-D-tagatofuranoside was as previously described (Wang, Z-H et al., 2021). The reaction of previously prepared 6-MOM-methyl-1,3-di-0-isopropylidene-α-D-tagatofuranoside with TsCl in pyridine afforded the corresponding 6-MOM-4-OTs-methyl-1,3-di-O-isopropylidene-α-D-tagatofuranoside (Scheme 3). Treatment of 6-MOM-methyl-1,3-di-O-isopropylidene-α-D-tagatofuranoside with DAST afforded the corresponding 6-MOM-4-F-methyl-1,3-di-O-isopropylidene-α-D-fructofuranoside (Wang, Z-H et al., 2021). Deprotection was carried out by brief heating in dioxane/diluted HCl to obtain [U¹³C]-4-fluoro-4-deoxyfructose in moderate yield.

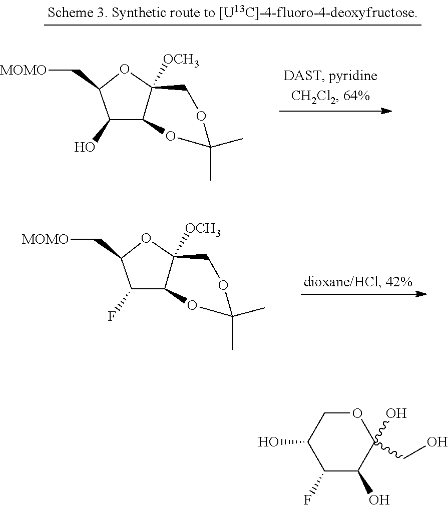

[UL-¹³C₆]-6-MOM-4-F-methyl-1,3-di-O-isopropylidene-α-D-fructofuranoside. A solution of [UL-¹³C₆]-6-MOM-methyl-1,3-di-O-isopropylidene-α-D-tagatofuranoside (106 mg, 0.37 mmol) in dry CH₂Cl₂ (7.5 mL) and pyridine (550 μL) was cooled to −40° C., while being continuously flushed with N₂ gas. Diethylaminosulfur trifluoride (DAST, 260 μL, 1.93 mmol) was added, the mixture was allowed to gradually warm up to rt and was stirred for 18 h at rt (N₂ atmosphere). The reaction was quenched by the addition of water (10 mL) and saturated NaHCO₃ solution (15 mL), the organic phase was separated, and the aqueous phase was extracted with CH₂Cl₂ (2×10 mL). Combined organic extract was dried and was concentrated; the residue was subjected to FCC on 40 g SiO₂, petroleum ether/EtOAc (7:3). Evaporation of the eluate afforded pale yellow oil, [UL-¹³C₆]-6-MOM-4-F-methyl-1,3-di-O-isopropylidene-α-D-fructofuranoside (65 mg, 61%). ¹H NMR (CDCl₃) δ 5.11-4.37 (m, 4H); 4.19-3.91 (m, 3H); 3.76-3.61 (m, 1H); 3.56-3.48 (m, 1H); 3.39 (s, 3H); 3.32 (d, J=4.0 Hz, 3H); 1.45 (s, 3H); 1.37 (s, 3H). ¹H{¹³C} NMR (CDCl₃) δ 4.84-4.65 (m, 3H); 4.41-4.28 (m, 1H); 4.24-4.15 (m, 1H); 3.91 (dd, J=14.5, 12.5 Hz, 2H); 3.81-3.66 (m, 2H); 3.39 (s, 3H); 3.32 (s, 3H); 1.45 (s, 3H); 1.36 (s, 3H). ¹H{¹⁹F} NMR (CDCl₃) δ 5.04-4.43 (m, 4H); 4.19-3.91 (m, 3H); 3.76-3.61 (m, 1H); 3.56-3.45 (m, 1H); 3.39 (s, 3H); 3.32 (d, J=4.0 Hz, 3H); 1.45 (s, 3H); 1.37 (s, 3H). ¹³C NMR (CDCl₃) δ 102.6-101.3 (m); 99.2-98.0 (m); 96.7-95.6 (m); 82.8-81.4 (m); 79.3-78.1 (m); 67.7-67.0 (m); 62.6-61.9 (m); 61.2 (m); 60.5 (m); 55.4 (s); 39.3 (m); 20.1 (s); 20.1 (s). ¹⁹F NMR (CDCl₃) δ −185.5 to −186.7 (m). ¹⁹F{¹H} NMR (CDCl₃) δ −185.6 to −186.5 (m). HRMS (ESI) m/z found 309.1427 [M+Na]⁺ (309.1421 calcd for ¹²C₆¹³C₆H₂₁FO₆Na).

[UL-¹³C₆]-4-F-D-fructose. 6 M HCl (300 μL) was added to a solution of [UL-¹³C₆]-6-MOM-4-F-methyl-1,3-di-O-isopropylidene-α-D-fructofuranoside (136 mg, 0.48 mmol) in dioxane (600 μL). The mixture was stirred for 15 min at 80° C., was cooled to 0° C. and the pH was adjusted to ˜6.5-7 (20% NaOH solution). The liquids were evaporated, and the residue was subjected to FCC on 20 g SiO₂, CH₂Cl₂/MeOH (9:1) later replaced with CH₂Cl₂/MeOH (4:1). Evaporation of the eluate afforded colorless oil; this was dissolved in small amount of water (˜4 ml), was transferred into a Falcon tube and was lyophilized to afford colorless semi-solid residue, [UL-¹³C₆]-4-F-D-fructose (48 mg, 54%). ¹H NMR (D₂O) δ 5.15-4.96 (m, 0.5H); 4.65-4.44 (m, 1H); 4.37-4.27 (m, 1H); 4.06-3.78 (m, 3H); 3.55-3.31 (m, 1.5H). ¹H{¹³C} NMR (D₂O) δ 4.92-4.71 (m, 1H); 4.28 (m, 1H); 4.13-4.03 (m, 2H); 3.81-3.73 (m, 2H); 3.58 (m, 1H). ¹H{¹⁹F} NMR (D₂O) δ 5.11-5.02 (m, 0.5H); 4.53 (m, 1H); 4.35-4.26 (m, 1H); 4.08-3.78 (m, 3H); 3.54-3.30 (m, 1.5H). ¹³C NMR (D₂O) δ 99.3-97.9 (m); 93.2-92.2 (m); 90.9-89.8 (m); 68.0-66.5 (m); 66.1-65.4 (m); 63.8-63.2 (m); 62.9-62.3 (m). ¹⁹F NMR (D₂O) δ −201.8 to −202.7 (m). ¹⁹F{¹H} NMR (D₂O) δ −201.8 to −202.7 (m). HRMS (ESI) m/z found 211.0699 [M+Na]⁺ (211.0690 calcd for ¹³C₆H₁₁FO₅Na).

### Example 4. Synthesis of [¹⁸F]-6-fluoro-6-deoxyfructose ([¹⁸F]6-FDF)

The synthesis of the cold standard, tosylated precursor and radiofluorinated [¹⁸F]6-FDF was performed as described previously (Sasa, K. et al., 2018). All characterization matched that described in the literature.

### Example 5. Synthesis of Cold Fluorination Standard ([¹⁹F]4-FDF)

Treatment of 6-MOM-methyl-1,3-di-O-isopropylidene-α-D-tagatofuranoside (Compound 4) with DAST afforded the corresponding 6-MOM-4-F-methyl-1,3-di-O-isopropylidene-α-D-fructofuranoside. Deprotection was carried out by brief heating in dioxane/diluted HCl to obtain 4-fluoro-4-deoxyfructose in moderate yield (Scheme 3).

### Example 6. Synthesis of 4-F-D-fructose

6-MOM-4-F-methyl-1,3-di-1-isopropylidene-α-D-fructofuranoside. A stirred solution of 6-MOM-methyl-1,3-di-O-isopropylidene-α-D-tagatofuranoside (Compound 4, 385 mg, 1.38 mmol) in dry CH₂Cl₂ (26 mL) and dry pyridine (2 mL) was cooled to −40° C. while being continuously flushed with N₂ gas. Diethylaminosulfur trifluoride (DAST, 760 μL, 5.81 mmol) was added over 1 minute period. The reaction mixture was allowed to gradually warm up to room temperature, while being stirred for 18 h (N₂ atmosphere). The reaction was quenched by the addition of water (30 mL) and saturated NaHCO₃ solution (40 mL). The organic phase was separated, and the aqueous phase was extracted with CH₂Cl₂ (2×30 mL). Combined organic extract was dried, was concentrated and the residue was subjected to FCC on 40 g SiO₂, petroleum ether/EtOAc (7:3). Evaporation of the eluate afforded pale yellow oil, 6-MOM-4-F-methyl-1,3-di-O-isopropylidene-α-D-fructofuranoside (250 mg, 64%), [α]_{D} +77.2 (c 0.60, CH₂Cl₂). ¹H NMR (CDCl₃) δ 4.83 (d, J=3.0 Hz, 0.5H); 4.69 (dd, J=7.5, 6.5 Hz, 2H); 4.66 (d, J=3.0 Hz, 0.5H); 4.39 (ddd, J=9.0, 6.0, 3.0 Hz, 0.5H); 4.31 (ddd, J=9.0, 6.0, 3.0 Hz, 0.5H); 4.22 (d, J=14.5 Hz, 1H); 3.92 (dd, J=13.5, 12.5 Hz, 2H); 3.74 (m, 2H); 3.39 (s, 3H); 3.32 (s, 3H); 1.46 (s, 3H); 1.37 (s, 3H). ¹H{¹⁹F} NMR (CDCl₃) δ 4.73 (m, 3H); 4.36 (m, 1H); 4.22 (m, 1H); 3.92 (dd, J=13.5, 12.5 Hz, 2H); 3.74 (m, 2H); 3.39 (s, 3H); 3.32 (s, 3H); 1.46 (s, 3H); 1.37 (s, 3H). ¹³C NMR (CDCl₃) δ 101.9 (d, J=1.5 Hz); 98.7 (d, J=24.0 Hz); 96.7, 96.1, 82.1 (d, J=27.0 Hz); 78.5 (d, J=27.0 Hz); 67.3 (d, J=7.5 Hz); 62.2, 55.3, 48.7, 27.2, 20.0. ¹⁹F NMR (CDCl₃) δ −185.9 (m). ¹⁹F{¹H} NMR (CDCl₃) δ −185.9 (s). HRMS (ESI) m/z found 303.1213 [M+Na]⁺ (303.1220 calcd for C₁₂H₂₁FO₆Na).

4-F-D-fructose. 6 M HCl (500 μL) was added to a stirred solution of 6-MOM-4-F-methyl-1,3-di-O-isopropylidene-α-D-fructofuranoside (210 mg, 0.75 mmol) in dioxane (1 mL). The mixture was stirred for 15 minutes at 80° C., was cooled to 0° C. and the pH was adjusted to ˜6-7 (20% NaOH solution). The solvent was evaporated, and the residue was subjected to FCC on 20 g SiO₂, CH₂Cl₂/MeOH (9:1), later replaced with CH₂Cl₂/MeOH (4:1). Evaporation of the eluate afforded colorless oil. This material was dissolved in small amount of water (˜5 mL), was transferred to a Falcon tube, was frozen and was lyophilized to afford colorless sticky solid, 4-fluoro-D-fructose (57 mg, 42%), [α]_{D} −105.1 (c 1.08, water), [α]_{D} −105.1 (c 1.08, water), [α]_{D} −116.0 (c 0.45, water)(Luo, Y. et al., 2022). ¹H NMR (D₂O) δ 4.85 (dd, J=9.5, 3.5 Hz, 0.5H); 4.68 (dd, J=9.5, 3.5 Hz, 0.5H); 4.24 (m, 1H); 4.03 (m, 2H); 3.72 (m, 2H); 3.53 (dd, J=11.5, 2.0 Hz, 1H). ¹H{¹⁹F} NMR (D₂O) δ 4.75 (m, 1H); 4.24 (m, 1H); 4.03 (m, 2H); 3.71 (m, 2H); 3.53 (d, J=11.5 Hz, 1H). ¹⁹F NMR (D₂O) δ −198.0 (m, minor); −202.2 (m, major); −202.8 (m, minor). ¹⁹F{¹H} NMR (D₂O) δ −198.0 (s, minor); −202.2 (s, major); −202.8 (s, minor). ¹H NMR (CD₃OD) δ 4.74 (dd, J=9.5, 3.5 Hz, 0.5H); 4.57 (dd, J=9.5, 3.5 Hz, 0.5H); 4.06 (m, 3H); 3.66 (m, 2H); 3.47 (m, 1H). ¹H{¹⁹F} NMR (D₂O) δ 4.66 (m, 1H); 4.06 (m, 3H); 3.67 (m, 2H); 3.47 (m, 1H). ¹³C NMR (CD₃OD) 5 104.0 (d, J=9.5 Hz, minor); 100.7 (minor); 99.9 (d, J=9.5 Hz, major); 99.7 (minor); 98.3 (minor); 97.3 (minor); 93.3 (d, J=181.0 Hz, major); 82.0 (d, J=25.5 Hz, minor); 81.4 (d, J=21.0 Hz, minor); 81.3 (d, J=25.0 Hz, minor); 75.9 (d, J=21.5 Hz, minor); 69.4 (d, J=16.5 Hz, major); 67.6 (d, J=18.0 Hz, major); 65.5 (major); 64.7 (minor); 64.0 (m, major+minor); 62.4 (d, J=4.5 Hz, minor). ¹⁹F NMR (CD₃OD) 5-191.4 (m, minor); −197.9 (m, minor); −204.1 (m, major). ¹⁹F{¹H} NMR (CD₃OD) 5-191.3 (s, minor); −197.9 (s, minor); −204.1 (s, major). HRMS (ESI) m/z found 205.0481 [M+Na]+(205.0488 calcd for C₆H₁₁FO₅Na).

### Example 7. Synthesis of 6-MOM-4-OTs-methyl-1,3-di-O-isopropylidene-α-D-tagatofuranoside (Compound 5) (Radiofluorination Precursor for [¹⁸F]4-FDF)

The preparation of 6-MOM-methyl-1,3-di-O-isopropylidene-α-D-tagatofuranoside (Compound 4) was as previously described (Wang, Z-H et al., 2021). The reaction of previously prepared 6-MOM-methyl-1,3-di-O-isopropylidene-α-D-tagatofuranoside (Compound 4) with TsCl in pyridine afforded the corresponding 6-MOM-4-OTs-methyl-1,3-di-O-isopropylidene-α-D-tagatofuranoside (Compound 5) (Scheme 4).

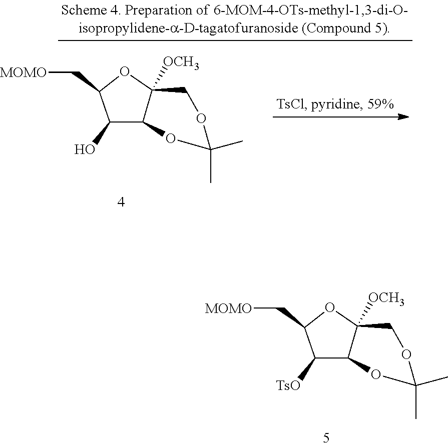

TsCl (167 mg, 0.88 mmol) was added to a solution of 6-MOM-methyl-1,3-di-O-isopropylidene-α-D-tagatofuranoside (Compound 4, 163 mg, 0.59 mmol) in dry pyridine (3 mL). The mixture was stirred for 18 h at rt and was partitioned between saturated NaHCO₃ solution (60 mL) and EtOAc (2×30 mL). Combined organic extract was dried and was concentrated, the residue was co-evaporated with toluene (˜100 mL), followed by FCC on 70 g SiO₂, petroleum ether/EtOAc (7:3). Evaporation of the eluate afforded colorless solid, 6-MOM-4-OTs-methyl-1,3-di-O-isopropylidene-α-D-tagatofuranoside (Compound 5, 149 mg, 59%), [α]_{D} +50.0 (c 0.87, CH₂Cl₂). ¹H NMR (CDCl₃) δ 7.82 (m, 2H); 7.34 (m, 2H); 5.29 (dd, J=8.0, 5.0 Hz, 1H); 4.66 (s, 2H); 4.43 (m, 1H); 3.80 (m, 5H); 3.37 (s, 3H); 3.22 (s, 3H); 2.45 (s, 3H); 1.30 (s, 3H); 1.17 (s, 3H). ¹³C NMR (CDCl₃) δ 145.0, 133.3, 129.7, 128.1, 99.9, 98.6, 96.6, 78.0, 77.2, 71.8, 67.3, 62.0, 55.2, 49.0, 27.7, 21.6, 19.2. HRMS (ESI) m/z found 455.1365 [M+Na]+(455.1352 calcd for C₁₉H₂₈O₉SNa).

### Example 8. Synthesis of [¹⁸F]-4-fluoro-4-deoxyfructose ([¹⁸F]4-FDF) (Compound 7)

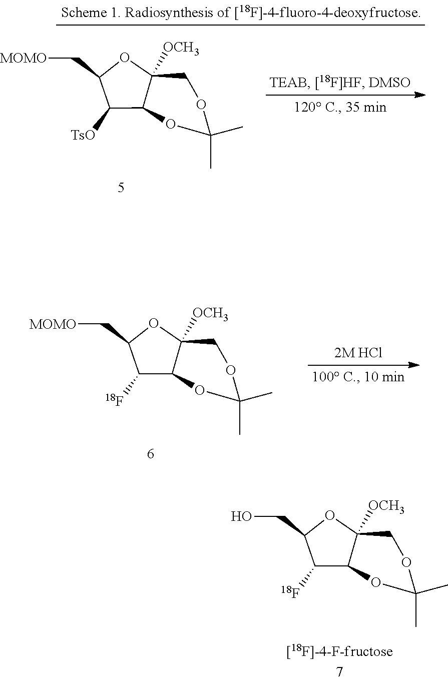

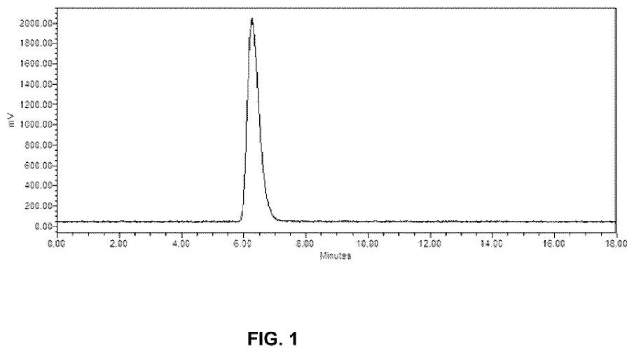

For each synthesis, conducted on a TRACERlab FX N Pro module from GE HealthCare, approx. 7500 MBq of [¹⁸F]HF (2.3 mL) were passed through a Sep-Pak Accell Plus QMA Plus Light Cartridge and consecutively eluted with triethylammonium bicarbonate (22.0 mg, 115 μmol) in water/acetonitrile (1:1/600 μL). The reactor was heated to 110° C. for 12 min, then acetonitrile (250 μL) added and heated to 110° C. for 3.5 min to azeotropically remove traces of water. Afterwards 6-MOM-4-OTs-methyl-1,3-di-O-isopropylidene-α-D-tagatofuranoside (7.0 mg, 16 μmol) in dimethyl sulfoxide (200 μL) was added and the mixture heated to 120° C. for 35 min. The reaction was then subjected to 2M hydrochloric acid (1.0 mL) and heated to 100° C. for 10 min. After diluting with water (1 mL) the crude mixture was pushed through a Sep-Pak Alumina N Plus Light Cartridge into a vial containing 0.1 M acetate buffer (2 mL, pH=5.4). Purification was performed via isocratic elution (1.5 mL/min) using 0.1 M acetate buffer (pH=5.4) as a mobile phase on a Luna C18 (250*10 mm, 10 μm) column. A peak eluting at ˜10-11 min was collected. Upon deprotection, the carbohydrate occurred as a mixture of pyranose and furanose form, two anomers for each form, hence the collected peak was broad, and shoulders might have been present. The decay corrected radiochemical yield ranged from 25-30%. Purity was confirmed by the UV/VIS and radio traces obtained through analytical HPLC on an apHera NH2 Polymer (150*4.6 mm, 5 μm) column. With a flow rate of 1 mL/min an initial mobile phase of acetonitrile/water (95:5) was over 5 min linearly changed to acetonitrile/water (5:95) and held for another 5 min, then over 2 min linearly changed to acetonitrile/water (95:5) and held for 5 min. FIG. 1 shows the analytical radio-HPLC trace after injection of 98 kBq of [¹⁸F]-4-fluoro-4-deoxyfructose in H₂O.

A reference for [¹⁸F]-6-MOM-4-fluoro-methyl-1,3-di-O-isopropylidene-α-D-fructofuranoside was obtained by skipping the deprotection step during the radio synthesis. A molar activity of ˜25.3±0.6 GBq/nmol was calculated with the prior determined molar attenuation coefficient after integration of the product peak in the analytical UV/VIS trace. Finally, the formulation for injection was done by adding saline (200 μL), 0.1 M sodium bicarbonate solution (50 μL) and sterile water (50 μL) per 37 MBq of product solution.

### Example 9. Fructolysis

The use of fructose as an energy source (i.e., fructolysis) occurs during the onset and progression of a variety of diseases, where inflammation-induced energy crises activate a fructolytic state in the affected tissues. In the heart, the switch from glycolysis to fructolysis has been identified in cardiac hypertrophy and myocardial infarction, with data supporting a hypoxia-driven activation of this aberrant metabolic program. In the brain, fructolysis is thought to be a putative driver of Alzheimer's disease, and has been shown to be pro-inflammatory with negative implications following traumatic injury, stroke injury, and psychological health. The switch from glucose to fructose as energy source may also be a key oncologic driver, promoting the progression of a variety of solid tumors through the concerted transcriptional activation of transport and metabolic machinery. Excessive fructose consumption has also been associated with a hepatic-centered metabolic syndrome thought to drive obesity and diabetes, and to be a major player in the related cardiovascular, ocular, and degenerative outcomes. The fundamental importance of fructolysis in a range of diseases has encouraged the development of methods to non-invasively map fructose metabolism, which remains a challenge.

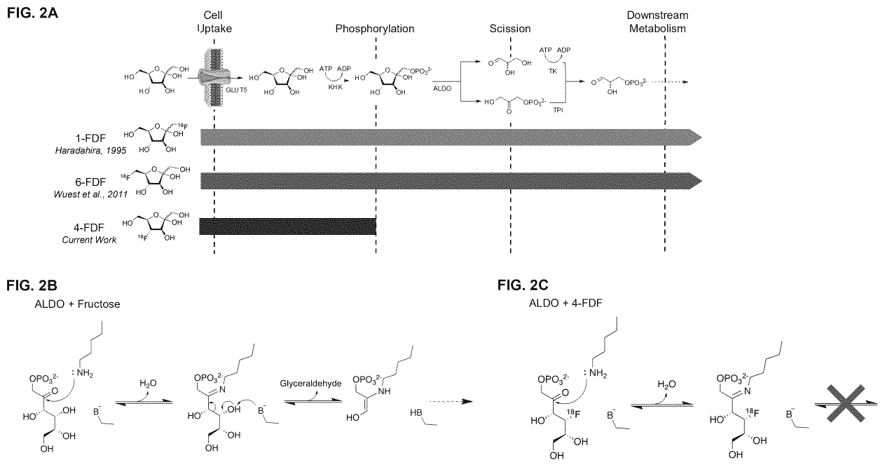

Canonical fructose metabolism, like that of glucose, begins with GLUT5-mediated transport into the cell and ketohexokinase-mediated trapping of the sugar as fructose-1-phosphate (FIG. 2A; Hannou, S. A. et al., 2018). Phosphorylation is followed by carbon chain scission through the activity of aldolase enzymes, and the subsequent formation of glyceraldehyde-3-phosphate that continues to be metabolized downstream. This metabolic cascade has been followed using non-invasive in vivo imaging in preclinical models, taking advantage of the spectroscopic capabilities of deuterium and hyperpolarized magnetic resonance imaging (Keshari, K. R. et al., 2009; Zhang, G. et al., 2023).

FIG. 2A depicts the initial metabolism of fructose comprises cell uptake, phosphorylation and scission steps mediated by GLUT5, ketohexokinase (KHK) and aldolase (ALDO), respectively. The progression of existing fructose-derived radiotracers, 1-FDF and 6-FDF, as well as the trapping of 4-FDF are shown.

Towards the clinical translational utilization of fructolysis as a quantitative imaging biomarker, previous work has attempted to trace fructose metabolism by positron emission tomography (PET) by installing radiofluorine (¹⁸F) at the C₁ or C₆ positions (Haradahira, T. et al., 1995; Wuest, M. et al., 2011; Wuest, M. et al., 2018; Bouvet, V. et al., 2014; Boyle, A. J. et al., 2022). The early metabolic trapping of fructose would lend itself to tracing of aberrant metabolism similarly to [¹⁸F]-2-fluoro-2-deoxy-D-glucose (FDG), the most extensively applied PET nuclear diagnostic used in the clinic. However, the significant bone-derived radioactivity observed by PET from previous radiodeoxyfluorofructose analogs suggest that cellular trapping was not achieved (FIG. 2A).

In redesigning a radiofluorinated fructose analog that results in trapping of the phosphorylated metabolite, the catalytic mechanism of aldolase, the enzyme for which fructose-1-phosphate is a substrate, was closely examined (FIG. 2B; Fushinobu, S. et al., 2011). Within the aldolase active site, the initial Schiff base formation with the C₂-carbonyl is immediately followed by a base-mediated proton abstraction from the C₄-hydroxyl moiety to induce C—C bond scission. FIG. 2B depicts the first two steps of the aldolase-mediated scission of fructose, where B⁻: basic residue in the aldolase active site residue.

Given the critical role of the C₄—OH in the catalytic mechanism, it was hypothesized that the deoxyfluorination of the C₄-position would prevent aldolase-mediated scission (FIG. 2C), resulting in the trapping of 4-deoxy-4-fluoro-D-fructose (4-FDF) within the metabolic cell of origin (FIG. 2A). The metabolic flux of 4-FDF was evaluated in vitro relative to 1- and 6-FDF, and the PET imaging of [¹⁸F]4-FDF was compared to [¹⁸F]6-FDF and [¹⁸F]FDG in tracing metabolism in mouse models of cancer and systemic inflammation. FIG. 2C depicts the proposed effect of C₄ deoxyfluorination on the aldolase mechanism, where B⁻: basic residue in the aldolase active site.

### Example 10. Characterization of the Structure-Activity Effect of Fructose Deoxyfluorination on Metabolic Flux

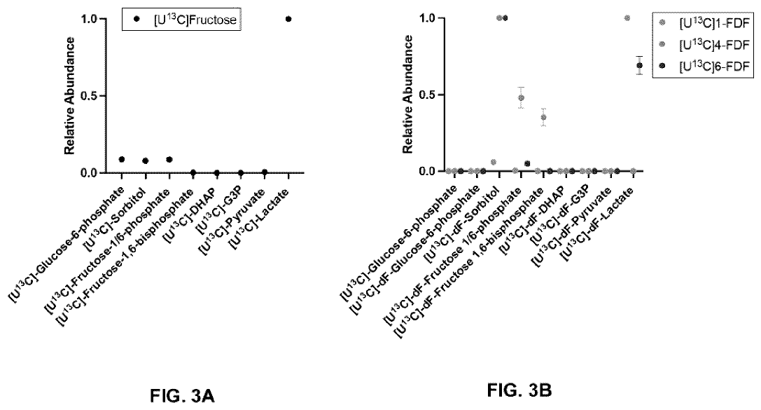

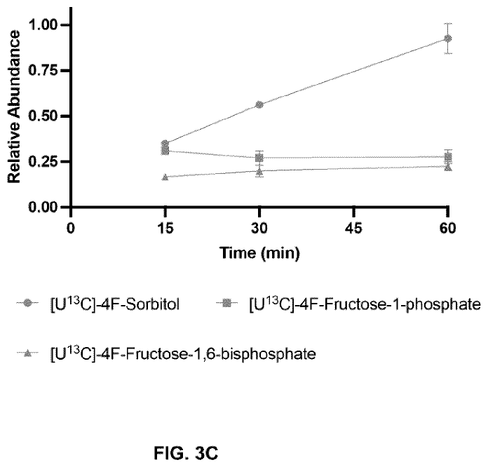

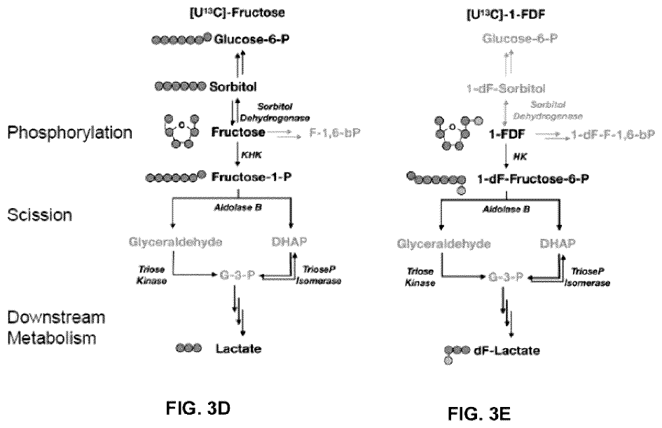

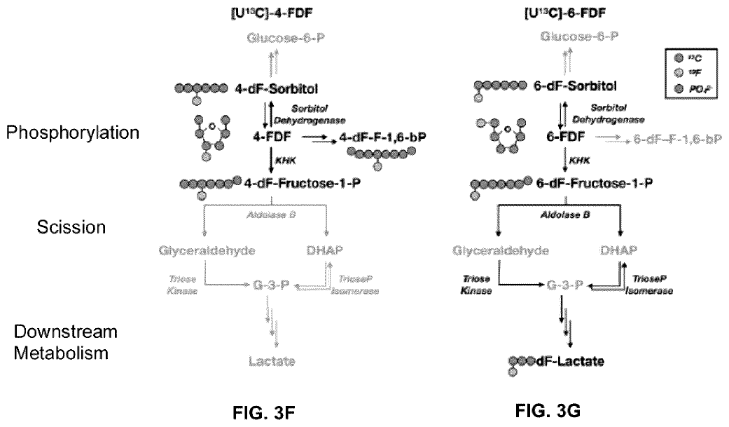

In order to characterize the structure-activity effect of fructose deoxyfluorination on metabolic flux, the metabolism of isotopically labelled [U¹³C]-fructose, and [U¹³C]-1-, [U¹³C]-6-, and [U¹³C]-4-FDF deoxyfluorinated fructose analogs was evaluated in vitro in HepG2 human hepatocarcinoma cells by mass spectrometry (FIGS. 3A-3G). HepG2 were chosen as a model cell line due to a recent report by Tee et al. outlining their propensity for fructolysis (Tee, S. S. et al., 2022). [U¹³C]-1- and [U¹³C]-6-FDF were synthesized according to previously published methods (Haradahira, T. et al., 1995; Wuest, M. et al., 2011), and [U¹³C]-4-FDF was synthesized as described hereinabove (Scheme 3).

After confirming that [U¹³C]-fructose was metabolized as expected through both fructolytic and polyol pathways to establish a baseline for tracing fructose metabolism (FIG. 3A), the relative flux of the deoxyfluorinated analogs was next examined (FIG. 3B). [U¹³C]-1-FDF showed limited metabolism through the polyol pathway, with the majority of the ¹³C-labelled cellular product being [U¹³C]-deoxyfluorolactate (FIG. 3B, light grey). Of critical importance, however, is that while [U¹³C]-6-FDF metabolism resulted in a substantial amount of [U¹³C]-deoxyfluorolactate production as a result of proceeding through scission and downstream metabolism (FIG. 3B, dark grey), [U¹³C]-4-FDF metabolism halted at [U¹³C]-4-fluorodeoxy-1-phosphate, the fructolytic metabolite that is the substrate for aldolase-mediated scission (FIG. 3B, medium grey). Uniquely, [U¹³C]-4-FDF metabolism also resulted in the accumulation of [U¹³C]-4-fluorodeoxyfructose-1,6-bisphophate. A key outcome of this experiment was the confirmation that all deoxyfluorinated analogs of fructose entered the cells rapidly (within 30 min). The relative abundance of metabolites from isotopically labelled [U¹³C]-fructose (FIG. 3A) and [U¹³C]-1- (FIG. 3B, light grey), [U¹³C]-4- (FIG. 3B, medium grey), and [U¹³C]-6-FDF (FIG. 3B, dark grey) are shown.

To validate the observed metabolite trapping, a time course evaluation of [U¹³C]-4-FDF metabolism was performed over 60 min, demonstrating the steady-state accumulation of [U¹³C]-4-fluorodeoxyfructose-1-phosphate and [U¹³C]-4-fluorodeoxyfructose-1,6-bisphosphate, and increase in [U¹³C]-4-fluorodeoxysorbitol throughout the 60 min of incubation (FIG. 3C). FIG. 3C shows time course of metabolite generation from [U¹³C]-4-FDF. The results of this study suggest that, like native fructose (FIG. 3D) neither [U¹³C]-1- (FIG. 3E) nor [U¹³C]-6-FDF are metabolically trapped (FIG. 3G). Rather, the deoxyfluorination of fructose at C₄ prevented aldolase-mediated hexose scission and trapped the deoxyfluorinated fructose analog in the cell (FIG. 3F). By rethinking the site of deoxyfluorination to afford metabolic trapping as informed by the catalytic mechanism of the enzyme immediately ensuing to the intended trapped metabolite, the chemical requirements for mapping fructolysis were uncovered. Metabolism schemes based on mass spectrometry results are shown for [U¹³C]-fructose (FIG. 3D), [U¹³C]-1- (FIG. 3E), [U¹³C]-4- (FIG. 3F), and [U¹³C]-6-FDF (FIG. 3G). Light grey: undetected metabolite or pathway; Black: detected metabolite or pathway; Blue circle: ¹³C, Yellow circle: ¹⁹F, Pink circle: PO₄²⁻.

### Example 11. Synthesis and Characterization of [¹⁸F]4-FDF

In order to proceed towards fructolysis mapping in vivo by PET, a radiodeoxyfluorination approach was designed to afford nucleophilic substitution at the C₄ position using standard radiochemical techniques somewhat related to the routine production of FDG. Details of the synthesis of compounds 1-4 have been reported previously (Suchy, M. et al., 2021), and further synthetic steps and chemical characterization for compounds 5, 6, and [¹⁸F]4-FDF (Compound 7) are provided hereinabove and are summarized in Scheme 6. The precursor synthesis began with C₁—OH methylation and dimethyl ketalation of C₂—OH and C₃—OH, followed by the protection of C₆—OH with chloromethyl methyl ether (MOMCl) in order to isolate the C₄—OH (Scheme 6A). The stereochemistry at C₄ was then inverted in two steps and was converted to the tosylated precursor 5 (Scheme 6A). The C₄-stereoinversion was necessary to allow the subsequent radiodeoxyfluorination step to restore the C₄-D-enantiomer after the [¹⁸F]TEAF-mediated nucleophilic attack (Scheme 6B, 6). Rapid on-module deprotection resulted in [¹⁸F]4-FDF (Compound 7) in good radiochemical yield (25-30%) and molar activity (25.3±0.6 GBq/nmol) comparable to that resulting from the routine production of FDG (Luurtsema, G. et al., 2021).

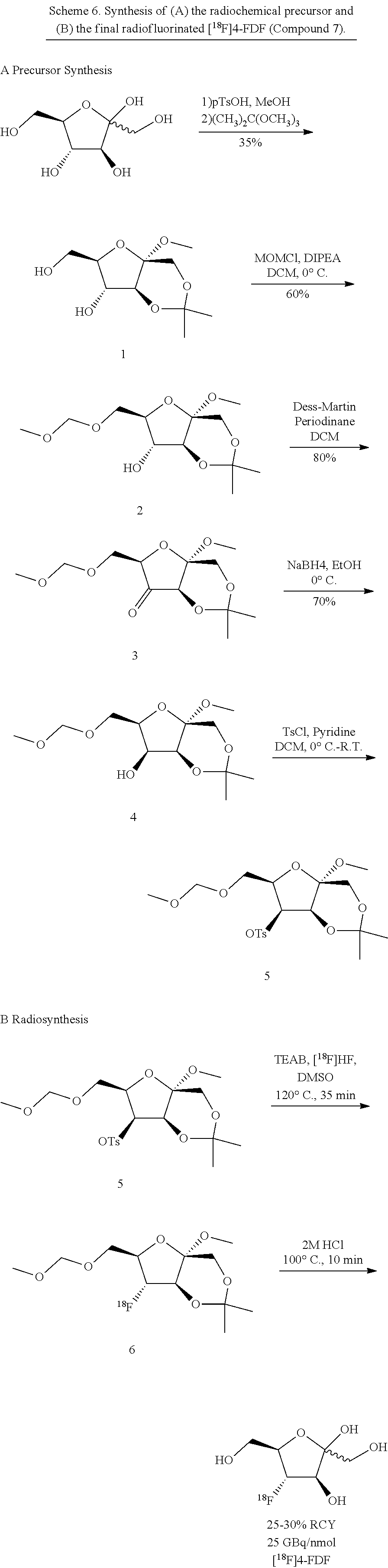

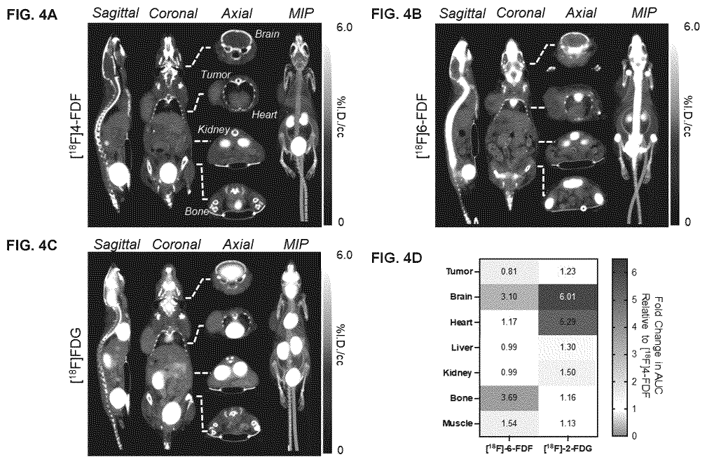

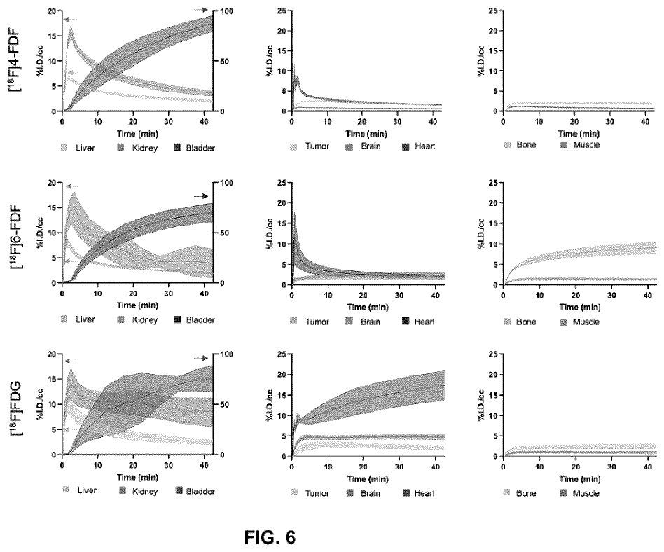

After the confirmation of cell uptake and intracellular trapping of [U¹³C]4-FDF, and the successful production of the radiofluorinated analog, the biodistribution of [¹⁸F]4-FDF was evaluated in a heterotopic HepG2 xenograft mouse model and compared to the biodistribution of [¹⁸F]6-FDF and [¹⁸F]FDG. PET/CT images shown are the summed images from 20-45 min after intravenous injection of (FIG. 4A) [¹⁸F]4-FDF, (FIG. 4B) [¹⁸F]6-FDF, or (FIG. 4C) [¹⁸F]FDG. Sagittal, coronal, and maximum intensity projection (MIP) are shown, in addition to axial sections at the level of the brain, heart, liver/kidneys, and hips. FIG. 4D shows the fold change in area under the curve (AUC) for the entire time-activity curve for [¹⁸F]6-FDF and [¹⁸F]FDG, normalized to the AUC values for [¹⁸F]4-FDF. Purple represents an increase in AUC and blue represents a decrease in AUC. The values are normalized fold-change in AUC.

[¹⁸F]1-FDF was not evaluated in vivo since it was already demonstrated to be poorly retained in cells in vitro and in vivo (Wuest, M. et al., 2018). Following intravenous injection, [¹⁸F]4-FDF was found to accumulate in the tumor, with renal exceeding hepatobiliary excretion (FIG. 4A). This pattern of radiotracer retention was similarly observed for [¹⁸F]6-FDF, with a key difference, however, being bone uptake (FIG. 4B). While any bone uptake was limited to <2% ID/g for [¹⁸F]4-FDF (FIGS. 4A and 6), bone uptake was 3.69-fold higher (>7% ID/g) following [¹⁸F]6-FDF imaging (FIGS. 4B and 40). This extensive bone uptake, which continued to increase overtime (FIG. 6), was reported previously for [¹⁸F]6-FDF (Wuest, M. et al., 2011), and is supported by the metabolic flux outcomes of [U¹³C]6-FDF demonstrating the production of [U¹³C]fluorodeoxylactate (FIGS. 4B and 4G).

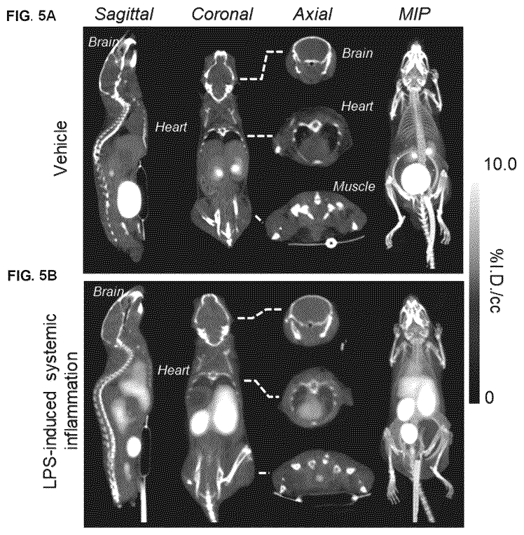

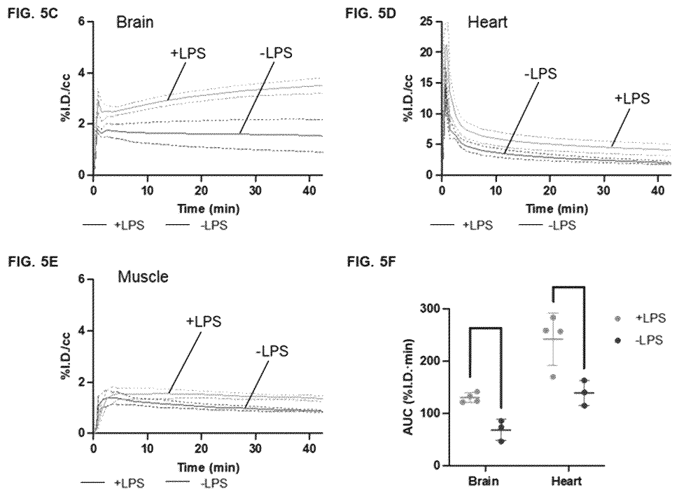

Overall, the accumulation of [¹⁸F]4-FDF in normal mouse tissues was lower than that observed with [¹⁸F]FDG (FIG. 4A vs. 4C). Notably, the area under the time activity curve (AUC) for [¹⁸F]FDG in the brain and heart was 6.01- and 5.29-fold greater, respectively, than for [¹⁸F]4-FDF (FIG. 40), suggesting that healthy brain and heart have a limited dependence on fructolysis for energy production. In order to further investigate whether a fructolytic switch occurs in inflammatory neural and cardiac tissues, a mouse model of systemic inflammation was examined. [¹⁸F]4-FDF PET/CT was performed on mice receiving vehicle (FIG. 5A) or bacterial cell wall LPS (FIG. 5B) 24 hr after injection. Sagittal, coronal, MIP, and axial sections of the brain, heart and muscle are shown. Time-activity curves (TAC) for brain (FIG. 5C), heart (FIG. 5D), and muscle (FIG. 5E) are shown for mice receiving vehicle (−LPS) or LPS (+LPS). Solid lines are the means and dotted lines are standard deviation. FIG. 5F shows comparison of TAC AUC values for brain and heart ROIs for mice receiving vehicle (−LPS) or LPS (+LPS). The plots show individual data points (circles), mean (long line) and s.d. (vertical line). * p<0.05 by ANOVA followed by Tukey's test.

Mice receiving saline vehicle (FIG. 5A) or intraperitoneal bacterial cell wall lipopolysaccharide (LPS), as previously described (FIG. 5B; Shrum, B. et al., 2014), were imaged by [¹⁸F]4-FDF PET/CT 24 hr after injection. A significant increase in cardiac (FIGS. 5D and 5F) and brain (FIGS. 5C and 5F) uptake of [¹⁸F]4-FDF was observed following LPS treatment in all mice evaluated. Both the brain and heart demonstrate inflammatory responses to LPS stimulation within 24 hr of its systemic introduction, mediated through toll-like receptor engagement on microglia or cardiac adrenergic cells (Yang, D. et al., 2022; Batista, C. R. A. et al., 2019). The low uptake of [¹⁸F]4-FDF in healthy brain and heart contributed to an increased signal-to-noise ratio for the mapping of cardio- and neuroinflammation in tissues that are otherwise highly glycolytic and have high [¹⁸F]FDG uptake in the absence of disease (FIG. 4C).

### Example 12. Dosimetry for [¹⁸F]-4-fluoro-4-deoxyfructose (Compound 7) Relative to [¹⁸F]-2-fluoro-2-deoxyglucose in Healthy Mice

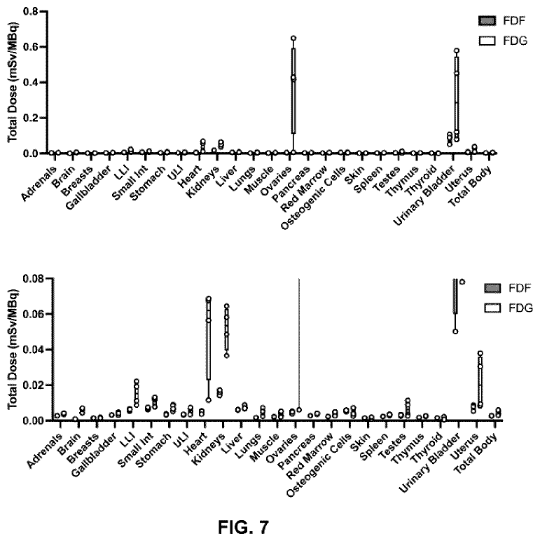

Image-based dosimetry was evaluated in Balb/C mice (n=4) using OLINDA software. Results are shown in FIG. 7. For all tissues, organ dose deposition from [¹⁸F]-4-fluoro-4-deoxyfructose (grey) was less than or equal to the dose deposition for [¹⁸F]-2-fluoro-2-deoxyglucose (white). The bottom plot is a zoom of the y-axis from the top plot.

In sum, taking a molecular design approach informed by the catalytic mechanism of aldolase, the fructolytic enzyme whose activity must be blocked in order to afford metabolic trapping, a novel radiodeoxyfluorinated analog of fructose was synthesized: [¹⁸F]4-FDF. Radiosynthesis was realised on a standard radiofluorination module in good yield and molar activity, mimicking the nucleophilic radiofluorination and acid-catalyzed deprotection used for the preparation of [¹⁸F]FDG (Scheme 1). As compared to previously reported C₁- and C₆-radioanalogs of fructose, using heavy isotope tracing by mass spectrometry, it was demonstrated that the C₄-deoxyfluorination of fructose led to trapping as fluorodeoxysorbitol and fluorodeoxyfructose-1-phosphate in vitro (FIG. 3). Key differences in polyol pathway flux were also observed between the different fluorinated positional isomers. The limited polyol flux observed for C₁-fluorodeoxyfructose was likely the result of improper substrate positioning in the sorbitol dehydrogenase active site by the deoxyfluorination of C₁, which prevents a critical C₁—OH-to-Zn interaction (Pauly, T. A. et al., Structure, 2003, 11: 1071-1085). In contrast, both [U¹³C]-6- and [U¹³C]-4-FDF were capable of proceeding through the polyol pathway but did not form detectable amounts of glucose-6-phosphate (FIG. 3B, dark grey and medium grey, respectively). The arrest at [U¹³C]-4/6-fluorodeoxysorbitol could be the result of the reduction of aldose reductase activity either through active site water displacement or catalytically detrimental interactions with the active site-adjacent specificity pocket (Rechlin, C. et al., ACS Chem. Biol. 2017, 12: 1397-1415; Sandner, A. et al., 2021). Notably, neither the C₁- nor C₆-fluorinated analogs led to trapping, but rather proceeded through fructolysis to produce fluorolactate. This result was recapitulated in vivo, where [¹⁸F]6-FDF showed significant bone uptake that was a result of metabolic processing, but which was not observed using [¹⁸F]4-FDF (FIG. 4). Both in vitro and in vivo data reported above demonstrate that radiodeoxyfluorination of fructose at C₄, but not C₆, can subvert cellular radiometabolite loss and bone accumulation.

Another important outcome of the stable tracing of fructolysis afforded by [¹⁸F]4-FDF was the observation of very low radiotracer uptake in healthy brain and heart (FIG. 5), tissues that are highly glycolytic and associated with high background levels of [¹⁸F]FDG uptake (FIG. 4). The low fructolytic background rates in these tissues afforded the sensitive mapping of the neuro- and cardioinflammatory response to systemic LPS administration by [¹⁸F]4-FDF (FIG. 5). Therefore, the aldolase-prescribed C₄-radiodeoxyfluorination of fructose resulted in radiotracer trapping upon intracellular uptake and phosphorylation (FIG. 2), overcoming limitations to fructolysis tracing by C₁- and C₆-radioanalogs and offering a clinically viable tool accessing fructolysis as an imaging diagnostic in tissues with a high baseline glycolytic index.

In conclusion, the metabolic flux of deoxyfluorofructose was characterized by heavy isotope labelling. [U¹³C]-1-FDF exhibited limited polyol metabolism, while both [U¹³C]-6-FDF and [U¹³C]-4-FDF showed polyol pathway involvement. Only [U¹³C]-4-FDF metabolism halted at [U¹³C]-4-fluorodeoxyfructose-1-phosphate, supporting its unique ability to be trapped within cells. [¹⁸F]4-FDF was synthesized with good molar activity and radiochemical yield. In a HepG2 xenograft mouse model, [¹⁸F]4-FDF exhibited tumor accumulation with minimal bone uptake, whereas [¹⁸F]6-FDF displayed substantial bone retention. [¹⁸F]4-FDF displayed lower accumulation in normal mouse tissues than [¹⁸F]FDG, notably in the brain and heart. As a result, a significant increase in [¹⁸F]4-FDF uptake in cardiac and brain tissues was observed after LPS treatment, highlighting the potential of [¹⁸F]4-FDF PET/CT for sensitive mapping of cardio- and neuroinflammation in highly glycolytic tissues. Overall, these results demonstrate the potential of [¹⁸F]4-FDF for mapping disease and/or injury involving cardio- and neuroinflammation.

### Example 13. Characterization of the Structure-Activity Effect of [¹⁸F]-4-fluoro-4-deoxytagatose on Fed and Fasted Mice

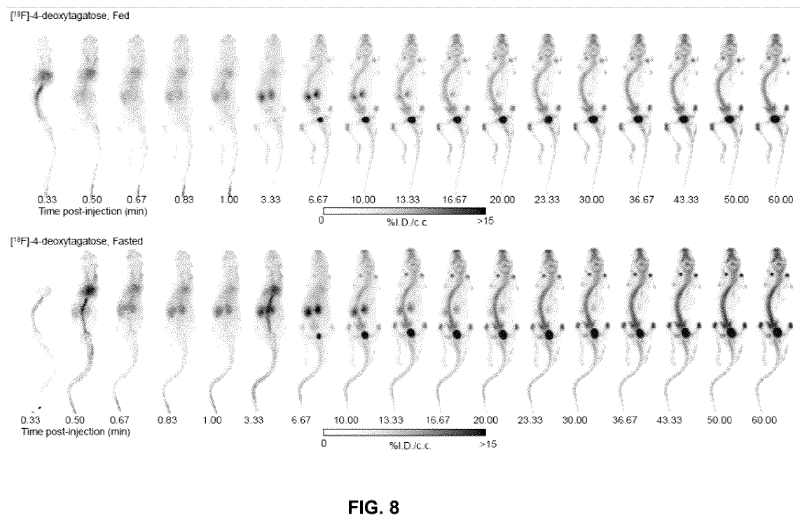

FIG. 8 shows time-course biodistribution of [¹⁸F]-4-fluoro-4-deoxytagatose in Balb/C mice freely fed (top row) or after overnight fasting (bottom row). Animals received ˜7.4 MBq i.v. immediately upon the initiation of image acquisition, which continued for 60 min. The dynamic scan was binned according to the times indicated. Regardless of fed or fasted state, [¹⁸F]-4-fluoro-4-deoxytagatose and/or one of its metabolites results in significant bone uptake.

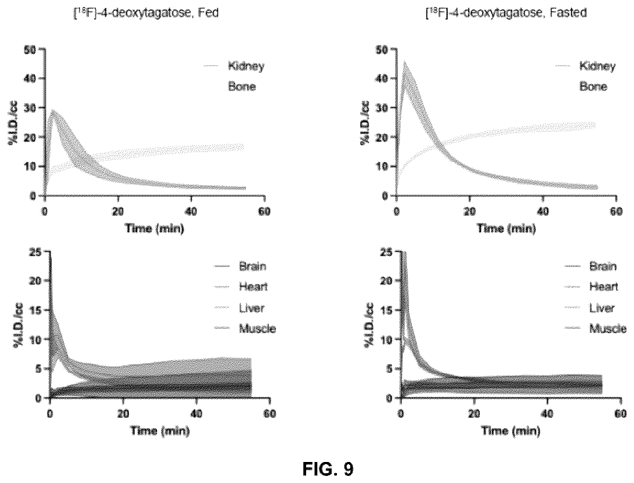

FIG. 9 shows TACs for [¹⁸F]-4-fluoro-4-deoxytagatose uptake in Balb/C mice freely fed (left) or after fasting (right) in the indicated tissues. Solid line=mean; Shaded region=standard deviation. Regions of interest were drawn over the tissues of interest, and the activity within that region of interest of known volume were plotted over the timecourse of the 60 min PET/CT acquisition. While the main route of clearance is the kidney, [¹⁸F]-4-fluoro-4-deoxytagatose and/or one of its metabolites results in significant bone uptake that is retained overtime.

### Example 14. Characterization of the Structure-Activity Effect of [¹⁸F]-4-fluoro-4-deoxyfructose on Fed and Fasted Mice

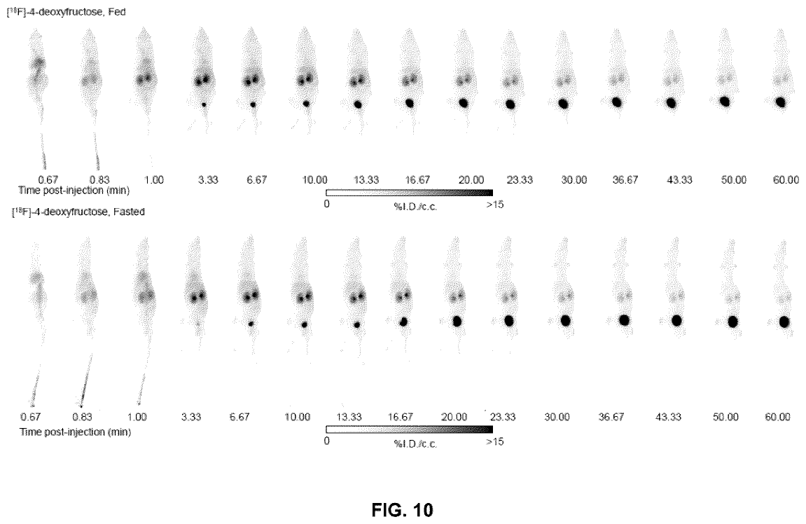

FIG. 10 shows time-course biodistribution of [¹⁸F]-4-fluoro-4-deoxyfructose in Balb/C mice freely fed (top row) or after overnight fasting (bottom row). Animals received ˜7.4 MBq i.v. immediately upon the initiation of image acquisition, which continued for 60 min. The dynamic scan was binned according to the times indicated. Regardless of fed or fasted state, there is limited bone uptake of [¹⁸F]-4-fluoro-4-deoxyfructose with the major clearance route being the kidneys. Additionally, the nutritional status of the animal has minimal impact on the biodistribution of the radiotracer.

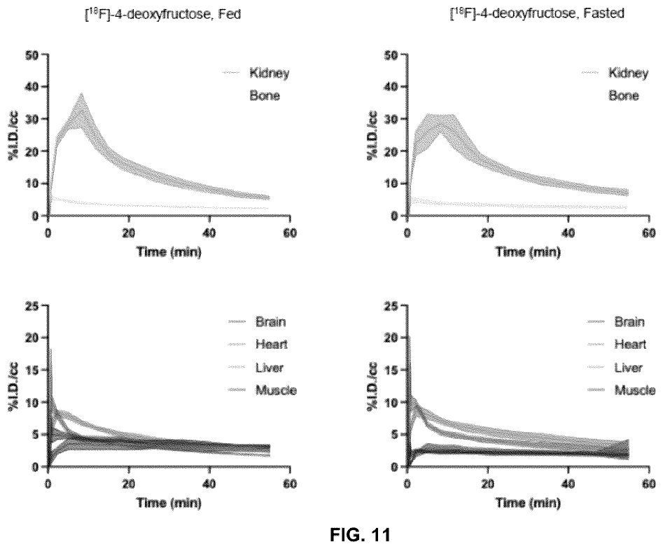

FIG. 11 shows TACs for [¹⁸F]-4-fluoro-4-deoxyfructose uptake in Balb/C mice freely fed (left) or after fasting (right) in the indicated tissues. Solid line=mean; Shaded region=standard deviation. Regions of interest were drawn over the tissues of interest, and the activity within that region of interest of known volume were plotted over the timecourse of the 60 min PET/CT acquisition. The main route of clearance is the kidney, and there is limited bone uptake of [¹⁸F]-4-fluoro-4-deoxyfructose overtime. The nutritional status of the animal has minimal impact on the biodistribution of the radiotracer.

It is clear from the differences observed in the biodistribution patterns following administration of [¹⁸F]-4-fluoro-4-deoxytagatose (Example 13) and [¹⁸F]-4-fluoro-4-deoxyfructose (Example 14) that the stereochemistry at C4 is critical to providing a radiotracer capable of mapping fructose metabolism. Radiolabeled tagatose, with its inverted stereochemistry at C4, does not exhibit the same biodistribution as the radiolabeled fructose, thus demonstrating that the stereochemistry of the fructose is essential to its specificity as a radiotracer suitable for imaging fructolysis in highly fructolytic tissues.

### Example 15. Characterization of the Structure-Activity Effect of [¹⁸F]-4-fluoro-4-deoxyfructose on Concussed Mice

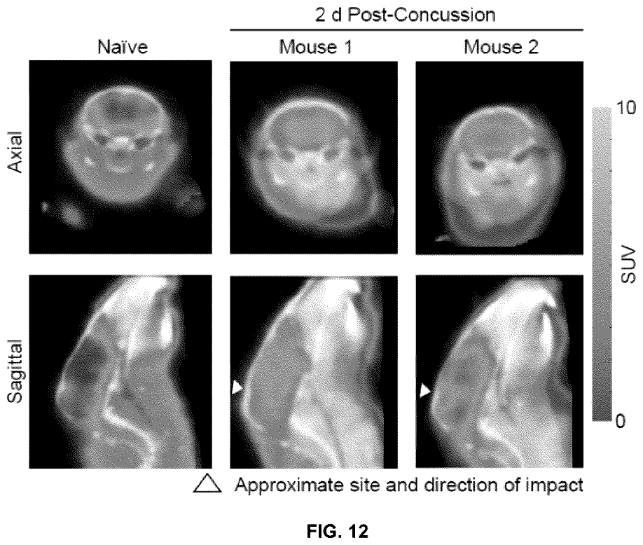

FIG. 12 Shows axial (top row) and sagittal (bottom row) PET/CT images of Balb/C mice receiving [¹⁸F]-4-fluoro-4-deoxyfructose, where the mice were either naïve (left column), or had received a concussive impact 48 h prior to imaging (middle and right columns). Arrowhead indicates approximate location and direction of concussive blow. Mice were either left without head impact (Naïve), or received a single closed-head impact vertically at bregma 48 hrs prior to imaging. Animals were imaged without fasting, and received ˜7.4 MBq i.v. immediately upon the initiation of image acquisition, which continued for 60 min. Average images from 20-40 min after the commencement of the scan are shown. While limited retention of [¹⁸F]-4-fluoro-4-deoxyfructose is noted in the naïve brain, enhanced radiotracer retention can be seen in both of the mice following concussion, especially below bregma where the impact was given.

### Experimental Procedures for the Examples

All reagents were commercially available. In D-[UL-¹³C₆]fructose analogs, all six carbon atoms were ¹³C enriched (99% enrichment level). All solvents were HPLC grade and used as such, except for water which was deionized (18.2 MΩ·cm⁻¹) and dichloromethane which was dried over Al₂O₃, in a solvent purification system. Organic extracts were dried with Na₂SO₄ and solvents were removed under reduced pressure in a rotary evaporator. Aqueous solutions were lyophilized. Flash column chromatography (FCC) was carried out using silica gel, mesh size 230-400 Å. Thin layer chromatography (TLC) was carried out on Al backed silica gel plates; compounds were visualized by orcinol stain. Specific rotations [α]_{D} were determined by polarimeter at ambient temperature using a 2 mL, 1 cm path length cell; the units are 10⁻¹ deg cm² g⁻¹ and the concentrations are reported in g/100 mL. NMR spectra were recorded on 300 MHz spectrometer; for ¹H (300 MHz), δ values were referenced as follows CDCl₃ (7.26 ppm); CD₃OD (3.31 ppm); D₂O (4.79 ppm) for ¹³C (75 MHz) CDCl₃ (77.0 ppm), CD₃OD (49.0 ppm). High resolution mass spectra (HR-MS) were obtained by electron spray ionization (ESI) time-of-flight (TOF) method.

Tracing of [U¹³C]-fructose metabolism in vitro. Human hepatocellular carcinoma cells (HepG2) were grown in Dulbecco's modified Eagle medium (DMEM) supplemented with 10% heat-inactivated fetal bovine serum (FBS) and 1% penicillin-streptomycin (P/S) until 80% confluent under cell culture conditions (37° C., 5% CO₂, humidified), at which point they were passaged. Cells were passaged three times before being seeded in to a 6-well plate and grown until 80% confluent. Supplemented media was aspirated and replaced with lactated Ringer's buffer (LRB) alone or containing 25 mM [U¹³C]-1-FDF, [U¹³C]-4-FDF, or [U¹³C]-6-FDF. Cells were incubated under cell culture conditions for 0, 15, 30, or 60 min. Following incubation, the plates were placed on ice and wells were aspirated. Wells were washed three times with ice-cold ammonium formate buffer (150 mM, pH 7.4) to remove unbound probe. A chilled (−20° C.) solution of 1:1 methanol:water was added to the wells and cells were scraped off of the wells into the solution. This solution was collected into bead-beater tubes containing 6 beads and stored at −80° C. until further analysis was performed.

Levels of fructose metabolites were assessed by liquid chromatography mass spectrometry (LC-MS). Sample temperature was maintained on ice or dry ice where possible, and all solvents were MS grade and pre-equilibrated to −20° C.

Metabolite extraction. Cell pellets were collected to a pre-chilled 2 mL tube containing 6 washed ceramic beads (1.4 mm). 380 μl of methanol:water (1:1) was added in the tube and kept in −80° C. until the day of metabolite extraction. On the day of extraction, samples were vortexed for 10 s and cell lysis was done by beating for 60 s at 2000 rpm (bead beating was done twice) after adding 220 μL of acetonitrile. Samples were then incubated with a 2:1 dichloromethane:water solution on ice for 10 minutes. The polar and non-polar phases were separated by centrifugation at 4000 g for 10 minutes at 1° C. The resulting upper phase, consisting of polar metabolites was dried with a refrigerated (−4° C.) centrivap concentrator (Labconco) and stored at −80° C. before LC-MS analyses.

LC-MS metabolite quantification. Samples to be injected in -ESI were re-suspended with 75% acetonitrile, cleared by centrifugation and run on an Agilent 6545B Q-TOF mass spectrometer equipped with a 1290 Infinity II ultra-high performance LC (Agilent Technologies). Continuous internal mass calibration was executed using signals from purine [12,000 full width at half maximum (FWHM) resolution] and hexakis (1H,1H,3H-tetrafluoropropoxy) phosphazine (24,000 FWHM resolution). All study samples were randomized before analysis and run using both high and low pH hydrophilic interaction chromatography (HILIC-Z) in negative ionization mode. HILIC separation was obtained using the Poroshell 120 HILIC-Z column (2.1 100 mm, 2.7 mm; Agilent) and corresponding guard column. The chromatographic conditions and mass spectrometry acquisition parameters were as described elsewhere (Zhu, Z. et al., 2023). Metabolite identification was confirmed by exact mass, retention time and subsequent MS/MS fragmentation for where standards are available. For the fluoro-fructose labelled metabolites, exact mass and MS/MS was considered for metabolite confirmation.

Animal Models. All animal research was approved by the IACUC of uOttawa under AUP SCe-3254-R3 (tumor study) and SCe-4019-A1 (inflammation study). Mice were housed in standard cages, kept on a 12-hr light/dark cycle and provided standard rodent chow and water ad libitum.

Eight-week-old female nu/nu mice were inoculated with 10×10⁶ HepG2 cells suspended in 50% Matrigel:DMEM subcutaneously under the left shoulder. Within 3-weeks of implantation, mice were imaged by PET/CT.

Eight-week-old male Balb/C mice received 5 mg/kg LPS through intraperitoneal injection 24-hr prior to planned PET/CT imaging. Mice were supported by being warmed, and by being given fluids subcutaneously 12 hr after LPS injection. The mice were monitored and scored for severity of response to LPS as described previously (Shrum, B. et al., 2014).

PET/CT Imaging. PET/CT imaging was carried out on a Bruker Si78PET/CT scanner with a 4-position hotel having adjustable isoflurane and respiratory monitoring for each position (Bruker USA). Tail veins were catheterized, and an anatomical CT was acquired over the whole of the mouse bodies using the “rat” settings. PET acquisition was started just prior to a bolus i.v. injection of approximately 200 μCi of radiotracer. Dynamic scans were acquired in list mode format over 45 min and sorted into 16×0.5-mm sinogram bins for image reconstruction (4×15 s, 4×60 s and 8×300 s). Iterative reconstruction was performed using 3D ordered-subsets expectation maximization (3D-OSEM) followed by fast maximum a posteriori (fastMAP) using Paravision 360 software v.3.4 (Bruker, USA). Four-mouse images were split into individual mice, and the bed was removed using PMOD v (Bruker, USA). VivoQuant v.2022 (InviCRO) was used for visualization of radioradiotracer uptake in tissues, to define the three-dimensional (3D) volumes of interest (VOI) and for 3D-visualisation to create volume rendering technique images. The count densities were averaged for all volumes of interest at each time point to obtain a time versus activity curve (TAC). Tumor and tissue TACs were normalized to injected dose, measured by a CRC-15 PET dose calibrator (Capintec, Inc., NJ, USA), and expressed as percentage injected dose per cubic centimeter of tissue (% ID/cc).

Statistical Analyses. Statistical analyses were performed using Prism (v. 9.5.0, GraphPad, Inc.). Comparisons across more than two groups were performed by one-way ANOVA followed by Tukey's test for honestly significant differences. Normality was assumed where appropriate for all data sets. Prior to ANOVA, Levene's test was used to confirm equal variance, and visual quantile-quantile plot analysis was used to confirm homoscedasticity.

### REFERENCES

Mirtschink P, Krishnan J, Grimm F, et al. HIF-driven SF3B1 induces KHK-C to enforce fructolysis and heart disease. Nature. 2015; 522(7557):444-449. doi:10.1038/nature14508.
Mirtschink P, Jang C, Arany Z, Krek W. Fructose metabolism, cardiometabolic risk, and the epidemic of coronary artery disease. Eur Heart J. 2018; 39(26):2497-2505. doi:10.1093/EURHEARTJ/EHX518.
Williams A L, Khadka V, Tang M, et al. HIF1 mediates a switch in pyruvate kinase isoforms after myocardial infarction. Physiol Genomics. 2018; 50(7):479-494. doi:10.1152/PHYSIOLGENOMICS.00130.2017/SUPPL_FILE/SUPPLEMENTAL
Johnson R J, Gomez-Pinilla F, Nagel M, et al. Cerebral Fructose Metabolism as a Potential Mechanism Driving Alzheimer's Disease. Front Aging Neurosci. 2020; 12:560865. doi:10.3389/FNAGI.2020.560865/BIBTEX.
Spagnuolo M S, Iossa S, Cigliano L. Sweet but Bitter: Focus on Fructose Impact on Brain Function in Rodent Models. Nutrients. 2021; 13(1):1-18. doi:10.3390/NU13010001.
Chen C, Zhang Z, Liu C, et al. ATF4-dependent fructolysis fuels growth of glioblastoma multiforme. Nat Commun 2022 131. 2022; 13(1):1-13. doi:10.1038/s41467-022-33859-9.
Liu H, Huang D, McArthur D L, Boros L G, Nissen N, Heaney A P. Fructose induces transketolase flux to promote pancreatic cancer growth. Cancer Res. 2010; 70(15):6368-6376. doi:10.1158/0008-5472.CAN-09-4615/656045/P/FRUCTOSE-INDUCES-TRANSKETOLASE-FLUX-TO-PROMOTE.
Helsley R N, Park S H, Vekaria H J, et al. Ketohexokinase-C regulates global protein acetylation to decrease carnitine palmitoyltransferase 1a-mediated fatty acid oxidation. J Hepatol. 2023; 79(1):25-42. doi:10.1016/j.jhep.2023.02.010.
Ward P S, Thompson C B. Metabolic Reprogramming: A Cancer Hallmark Even Warburg Did Not Anticipate. Cancer Cell. 2012; 21(3):297-308. doi:10.1016/J.CCR.2012.02.014.
Song A, Mao Y, Wei H. GLUT5: structure, functions, diseases and potential applications. Acta Biochim Biophys Sin (Shanghai). 2023; Advanced 0:10.3724/abbs.2023158. doi:10.3724/abbs.2023158.
Hannou S A, Haslam D E, McKeown N M, Herman M A. Fructose metabolism and metabolic disease. J Clin Invest. 2018; 128(2):545. doi:10.1172/JC196702.
Kolderup A, Svihus B. Fructose Metabolism and Relation to Atherosclerosis, Type 2 Diabetes, and Obesity. J Nutr Metab. 2015; 2015:823081. doi:10.1155/2015/823081.
Harder J M, Guymer C, Wood J P M, et al. Disturbed glucose and pyruvate metabolism in glaucoma with neuroprotection by pyruvate or rapamycin. Proc Natl Acad Sci USA. 2020; 117(52):33619-33627. doi:10.1073/PNAS.2014213117.
Keshari K R, Wilson D M, Chen A P, et al. Hyperpolarized [2-13C]-fructose: A hemiketal DNP substrate for in vivo metabolic imaging. J Am Chem Soc. 2009; 131(48):17591-17596. doi:10.1021/JA9049355/SUPPL_FILE/JA9049355_SI_001.PDF.
Zhang G, Cullen Q, Berishaj M, Deh K, Kim N, Keshari K R. [6,6′-2H2] fructose as a deuterium metabolic imaging probe in liver cancer. NMR Biomed. 2023; 36(10):e4989. doi:10.1002/nbm.4989.
Haradahira T, Tanaka A, Maeda M, Kanazawa Y, Ichiya Y I, Masuda K. Radiosynthesis, rodent biodistribution, and metabolism of 1-deoxy-1-[18F]fluoro-d-fructose. Nucl Med Biol. 1995; 22(6):719-725. doi:10.1016/0969-8051(95)00018-S.
Wuest M, Trayner B J, Grant T N, et al. Radiopharmacological evaluation of 6-deoxy-6-[18F]fluoro-D-fructose as a radiotracer for PET imaging of GLUT5 in breast cancer. Nucl Med Biol. 2011; 38(4):461-475. doi:10.1016/J.NUCMEDBIO.2010.11.004.
Wuest M, Hamann I, Bouvet V, et al. Molecular Imaging of GLUT1 and GLUT5 in Breast Cancer: A Multitracer Positron Emission Tomography Imaging Study in Mice. Mol Pharmacol. 2018; 93(2):79-89. doi:10.1124/MOL.117.110007.
Bouvet V, Jans H S, Wuest M, et al. Automated synthesis and dosimetry of 6-deoxy-6-[18F]fluoro-D-fructose (6-[18F]FDF): a radiotracer for imaging of GLUT5 in breast cancer. Am J Nucl Med Mol Imaging. 2014; 4(3):248. Accessed Oct. 1, 2023. /pmc/articles/PMC3999405/.
Boyle A J, Murrell E, Tong J, et al. PET Imaging of Fructose Metabolism in a Rodent Model of Neuroinflammation with 6-[18F]fluoro-6-deoxy-D-fructose. Molecules. 2022; 27(23):8529. doi:10.3390/MOLECULES27238529/S1.
Fushinobu S, Nishimasu H, Hattori D, Song H J, Wakagi T. Structural basis for the bifunctionality of fructose-1,6-bisphosphate aldolase/phosphatase. Nature. 2011; 478(7370):538-541. doi:10.1038/nature10457.
Trayner B J, Grant T N, West F G, Cheeseman C I. Synthesis and characterization of 6-deoxy-6-fluoro-d-fructose as a potential compound for imaging breast cancer with PET. Bioorg Med Chem. 2009; 17(15):5488-5495. doi:10.1016/J.BMC.2009.06.034.
Menzies C, Naz S, Patten D, Lacoste B, Alquier T, Bennett B M. Distinct basal metabolism in three mouse models of neurodevelopmental disorders. eNeuro. 2021; 8(2):ENEURO.0292-20.2021. doi:10.1523/ENEURO.0292-20.2021.
Shrum B, Anantha R V., Xu S X, et al. A robust scoring system to evaluate sepsis severity in an animal model. BMC Res Notes. 2014; 7(1):1-11. doi:10.1186/1756-0500-7-233/FIGURES/4.
Tee S S, Kim N, Cullen Q, et al. Ketohexokinase-mediated fructose metabolism is lost in hepatocellular carcinoma and can be leveraged for metabolic imaging. Sci Adv. 2022; 8(14):eabm7985. doi:10.1126/SCIADV.ABM7985.
Suchy M, Charlton T A, Ben R N, Shuhendler A J. Synthesis of natural/13C-nriched d-tagatose from natural/13C-enriched d-fructose. Carbohydr Res. 2021; 507:108377. doi:10.1016/J.CARRES.2021.108377.
Luurtsema G, Pichler V, Bongarzone S, et al. EANM guideline for harmonisation on molar activity or specific activity of radiopharmaceuticals: impact on safety and imaging quality. EJNMMI Radiopharm Chem. 2021; 6(1):1-16. doi:10.1186/S41181-021-00149-6/TABLES/1.
Yang D, Dai X, Xing Y, et al. Intrinsic cardiac adrenergic cells contribute to LPS-induced myocardial dysfunction. Commun Biol. 2022; 5(1):96. doi:10.1038/s42003-022-03007-6.
Batista C R A, Gomes G F, Candelario-Jalil E, Fiebich B L, de Oliveira A C P. Lipopolysaccharide-induced neuroinflammation as a bridge to understand neurodegeneration. Int J Mol Sci. 2019; 20(9):2293. doi:10.3390/ijms20092293.
Pauly T A, Ekstrom J L, Beebe D A, et al. X-Ray Crystallographic and Kinetic Studies of Human Sorbitol Dehydrogenase. Structure. 2003; 11(9):1071-1085. doi:10.1016/S0969-2126(03)00167-9.
Rechlin C, Scheer F, Terwesten F, et al. Price for Opening the Transient Specificity Pocket in Human Aldose Reductase upon Ligand Binding: Structural, Thermodynamic, Kinetic, and Computational Analysis. ACS Chem Biol. 2017; 12(5):1397-1415. doi:10.1021/ACSCHEMBIO.7B00062/SUPPL_FILE/CB7B00062_SI_002.ZIP.
Sandner A, Ngo K, Sager C P, et al. Which properties allow ligands to open and bind to the transient binding pocket of human aldose reductase? Biomolecules. 2021; 11(12):1837. doi:10.3390/BIOM11121837/S1.
Pérez-Tomás R, Pérez-Guillén I. Lactate in the tumor microenvironment: An essential molecule in cancer progression and treatment. Cancers (Basel). 2020; 12(11):1-29. doi:10.3390/cancers12113244.
Wang Z-H, Peng W-B, Zhang P, Yang X-P, Zhou Q. Lactate in the tumour microenvironment: From immune modulation to therapy. EBioMedicine. 2021; 73:103627. doi:10.1016/j.ebiom.2021.103627.
Sasa K, Yoshimura K, Yamada A, et al. Monocarboxylate transporter-1 promotes osteoblast differentiation via suppression of p53, a negative regulator of osteoblast differentiation. Sci Rep. 2018; 8(1):10579. doi:10.1038/s41598-018-28605-5.
Luo Y, Gou H, Chen X, Li L, Wang X, Xu Y. Lactate inhibits osteogenic differentiation of human periodontal ligament stem cells via autophagy through the MCT1-mTOR signaling pathway. Bone. 2022; 162:116444. doi:10.1016/j.bone.2022.116444.
Zhu Z, Chen Y, Zou J, et al. Lactate Mediates the Bone Anabolic Effect of High-Intensity Interval Training by Inducing Osteoblast Differentiation. J Bone Jt Surg. 2023; 105(5):369-379. doi:10.2106/JBJS.22.01028.
Rabinowitz J D, Enerbsck S. Lactate: the ugly duckling of energy metabolism. Nat Metab. 2020; 2(7):566-571. doi:10.1038/s42255-020-0243-4.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

The contents of all documents and references cited herein are hereby incorporated by reference in their entirety.

1. A compound of Formula (I), or a tautomer or anomer thereof, or a pharmaceutically acceptable salt thereof: 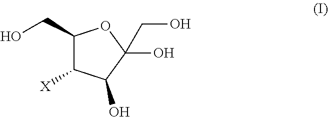
wherein X is ¹⁸F, ¹¹C, ⁷⁵Br, ⁷⁷Br, ¹²⁴I, ¹³¹I, or ²¹¹At.

2. The compound of claim 1, wherein the compound is: 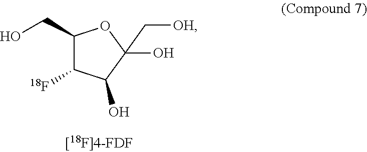 [SMILES: OC[C@H]1OC(O)(CO)[C@@H](O)[C@@H]1[18F]]
or a tautomer or anomer thereof.

3. A composition comprising the compound of claim 2, and a pharmaceutically acceptable carrier or excipient.

4. A compound of Formula (IIa) or (IIa′), or a pharmaceutically acceptable salt or ester thereof: 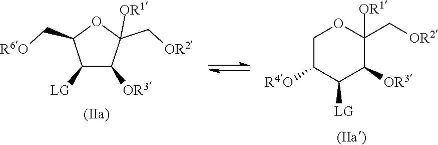
wherein:
R¹, R^{2′}, R^{3′}, R^{4′} and R^{6′} are each independently selected from —C(═O)(lower alkyl) or —C(═O)Ph; and
LG is 4-methylphenylsulfonyl (—OTs), methanesulfonyl (—OMs), trifluoromethanesulfonyl (—OTf), 4-nitrophenylsulfonyl (—ONs), phenylsulfonyl, —Br, —Cl, or —I.

5. The compound claim 4, wherein R^{1′}, R^{2′}, R^{3′}, R^{4′} and R^{6′} are each —C(═O)Ph.

6. The compound of claim 4, wherein LG is —OTs.

7. The compound of claim 4, wherein the compound is

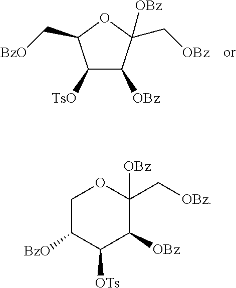

8. A compound of formula (IIb) or (IIb′), or a pharmaceutically acceptable salt or ester thereof: 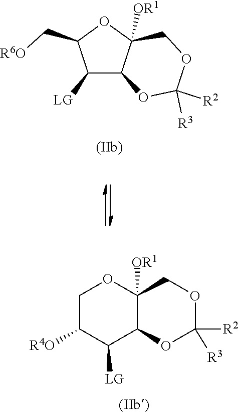
wherein:
R¹ is lower alkyl;
R² is H and R³ is phenyl, or R² and R³ are each independently lower alkyl;
R⁴ and R⁶ are methoxymethyl (MOM), t-butyl (t-Bu), benzyl (Bn), trimethylsilyl (TMS), t-butyldimethylsilyl (TBDMS), or tetrahydropyranyl (THP); and
LG is 4-methylphenylsulfonyl (—OTs), methanesulfonyl (—OMs), trifluoromethanesulfonyl (—OTf), 4-nitrophenylsulfonyl (—ONs), phenylsulfonyl, —Br, —Cl, or —I.

9. The compound of claim 8, wherein R¹, R² and R³ are each methyl.

10. The compound of claim 8, wherein R⁴ is MOM.

11. The compound of claim 8, wherein LG is —OTs.

12. The compound of claim 8, wherein the compound is

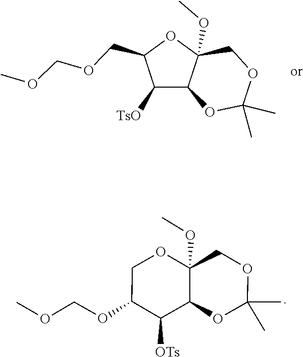

13-24. (canceled)

25. A method of biomedical imaging, comprising administering the compound of claim 2 to a subject, and imaging one or more tissues in the subject using PET (positron emission tomography), PET/CT (PET in conjunction with computed tomography), or PET/MRI (PET in conjunction with magnetic resonance imaging) to measure accumulation of Compound 7 or a tautomer or anomer thereof, and their metabolites in the tissue.

26. The method of claim 25, wherein the metabolites are fructolytic metabolites and the step of imaging identifies the presence or measures the amount of fructolysis in the one or more tissues to diagnose or monitor a fructolysis-associated disorder in the subject.

27.-50. (canceled)

51. The method of claim 26, wherein the fructolysis-associated disorder is cardiac hypertrophy, myocardial infarction, a cardiovascular disease, a neurodegenerative disease, an ocular disease, a traumatic injury, a stroke injury, a cancer, metabolic syndrome, obesity, diabetes, or inflammation.

52. The method of claim 51, wherein the neurodegenerative disease is Alzheimer's disease.

53. The method of claim 51, wherein the traumatic injury is traumatic brain injury.

54. The method of claim 51, wherein the inflammation is systemic inflammation.

55. The method of claim 51, wherein the inflammation is cardio- and/or neuroinflammation.

56. The method of claim 51, wherein the cancer is a solid tumor.

57. A composition comprising the compound of claim 1 and a pharmaceutically acceptable carrier or excipient.
